SEQUENCE LISTING (2 sequences)

SEQ ID NO: 1 - DNA, 50 bp, Artificial Sequence
  16S_V3_F
  misc_feature (42)..(42): any nucleotide
tcgtcggcagcgtcagatgtgtataagagacagcctacgggnggcwgcag

SEQ ID NO: 2 - DNA, 55 bp, Artificial Sequence
  16S_V4_R
gtctcgtgggctcggagatgtgtataagagacaggactachvgggtatctaatcc

(19) United States
(12) Patent Application Publication
(10) Pub. No.: US 2020/0157608 A1
(43) Pub. Date: May 21, 2020

(54) METHOD FOR DIAGNOSING LUNG CANCER VIA BACTERIAL METAGENOMIC ANALYSIS

(71) Applicant: MD HEALTHCARE INC., Seoul (KR)

(72) Inventor: Yoon-Keun Kim, Gyeonggi-do (KR)

(21) Appl. No.: 16/473,020

(22) PCT Filed: Dec. 21, 2017
(86) PCT No.: PCT/KR2017/015173

§ 371 Date: Jun. 24, 2019

(30) Foreign Application Priority Data
Dec. 26, 2016 (KR) 10-2016-0179225
Jun. 27, 2017 (KR) 10-2017-0080905
Jun. 27, 2017 (KR) 10-2017-0080906

(51) Int. Cl. C12Q 1/689; C12Q 1/6886; C12Q 1/686; C12Q 1/6806

(52) U.S. Cl. CPC C12Q 1/689; C12Q 1/6806; C12Q 1/686; C12Q 1/6886

(57) ABSTRACT

The present invention relates to a method for diagnosing lung cancer via bacterial metagenomic analysis and, more particularly, a method for diagnosing lung cancer by analyzing an increase or decrease in content of extracellular vesicles derived from specific bacteria through bacterial metagenomic analysis using a subject-derived sample. An extracellular vesicle secreted from a bacterium present in the environment can be absorbed into the body and directly affect the occurrence of cancer, and lung cancer is difficult to diagnose early on before any symptom appears, which makes efficient treatment difficult. As such, through the metagenomic analysis on a gene present in a bacterium-derived extracellular vesicle using a human body-derived sample according to the present invention, the risk of the onset of lung cancer can be predicted in advance, enabling early diagnosis and prediction of a lung cancer risk group and delay of the time of the onset or prevention of the onset with proper care, and early diagnosis is still possible even after the onset, which can lower the incidence rate of lung cancer and enhance the treatment effect.

### TECHNICAL FIELD

The present invention relates to a method of diagnosing lung cancer through bacterial metagenomic analysis, and more particularly, to a method of diagnosing lung cancer by analyzing an increase or decrease in content of extracellular vesicles derived from specific bacteria by bacterial metagenomic analysis using a subject-derived sample.

### BACKGROUND ART

Lung cancer is a malignant tumor originating from the lungs, and, in spite of the development of modern medical science, lung cancer has a 5-year survival rate of less than 50% and is the leading cause of death in Korea. Lung cancer is broadly classified into small cell lung cancer and non-small cell lung cancer according to histological type, and the small cell lung cancer has a feature distinctively distinguished from other types of lung cancer in terms of treatment and prognosis, and thus histological examination results of lung cancer are very important in determining a decision for therapeutic policy.

Lung cancer has symptoms such as coughing, hemoptysis, chest pain, dyspnea, and the like. However, when such symptoms are shown, there are many cases where lung cancer has already progressed, and frequent cases where there are no symptoms yet lung cancer is progressing, and only 5% to 15% of lung cancer patients are diagnosed when they have no symptoms and most lung cancer patients are diagnosed with lung cancer after experiencing such symptoms. So far, there has been no certified screening method for early detection of lung cancer before symptoms are shown, early detection of lung cancer using chest computed tomography (CT) is most commonly used, but the effectiveness thereof has yet been proved. Thus, there is an urgent need to develop a method of enhancing therapeutic efficiency by early detection of lung cancer, and, prior to the development of such method, it is very important to differentiate methods for early diagnosis and treatment by predicting the risk for lung cancer, and thus research into and development of these methods are required.

Meanwhile, it is known that the number of microorganisms symbiotically living in the human body is 100 trillion which is 10 times the number of human cells, and the number of genes of microorganisms exceeds 100 times the number of human genes. A microbiota or microbiome is a microbial community that includes bacteria, archaea, and eukaryotes present in a given habitat, and intestinal microbiota is known to play a vital role in human's physiological phenomena and significantly affect human health and diseases through interactions with human cells. Bacteria symbiotically living in the human body secrete nanometer-sized vesicles to exchange information about genes, proteins, and the like with other cells. The mucous membranes form a physical barrier membrane that does not allow particles with the size of 200 nm or more to pass therethrough, and thus bacteria symbiotically living in the mucous membranes are unable to pass therethrough, but bacterial-derived vesicles have a size of approximately 100 nm or less and thus relatively freely pass through the mucous membranes and are absorbed into the human body.

With regard to the onset of lung cancer, chronic obstructive pulmonary disease (COPD) is a vital risk factor, and in clinical studies, it has been reported that COPD itself is a vital risk factor for the onset of lung cancer regardless of smoking. In addition, the fact that the onset of lung cancer has recently increased in non-smokers suggests that there is a causative factor for the onset of lung cancer other than smoking.

Metagenomics, also called environmental genomics, may be analytics for metagenomic data obtained from samples collected from the environment, and collectively refers to a total genome of all microbiota in the natural environment in which microorganisms exist and was first used by Jo Handelsman in 1998 (Handelsman et al., 1998 Chem. Biol. 5, R245-249). Recently, the bacterial composition of human microbiota has been listed using a method based on 16s ribosomal RNA (16s rRNA) base sequences, and 16s ribosomal RNA analyzes sequences using a 454FLX titanium platform. In the onset of lung cancer, however, identification of causative factors of lung cancer through metagenomic analysis of bacteria-derived vesicles isolated from a human-derived substance, such as blood or the like, and a method of predicting lung cancer have never been reported.

### DISCLOSURE

### Technical Problem

To diagnose lung cancer based on causative factors thereof, the inventors of the present invention extracted DNA from bacteria-derived extracellular vesicles isolated from blood, which is a subject-derived sample, and performed metagenomic analysis on the extracted DNA, and, as a result, identified bacteria-derived extracellular vesicles capable of acting as a causative factor of lung cancer, thus completing the present invention.

Therefore, it is an object of the present invention to provide a method of providing information for lung cancer diagnosis by metagenomic analysis of bacteria-derived extracellular vesicles.

However, the technical goals of the present invention are not limited to the aforementioned goals, and other unmentioned technical goals will be clearly understood by those of ordinary skill in the art from the following description.

### Technical Solution

According to an aspect of the present invention, there is provided a method of providing information for lung cancer diagnosis, comprising the following processes:

(a) extracting DNA from extracellular vesicles isolated from a subject sample;

(b) performing polymerase chain reaction (PCR) on the extracted DNA using a pair of primers having SEQ ID NO:1 and SEQ ID NO: 2; and

(c) comparing an increase or decrease in content of bacteria-derived extracellular vesicles of the subject sample with that of a normal individual-derived sample, a chronic obstructive pulmonary disease patient-derived sample, or an asthma patient-derived sample through sequencing of a product of the PCR.

The present invention also provides a method of diagnosing lung cancer, comprising the following processes:

(a) extracting DNA from extracellular vesicles isolated from a subject sample;

(b) performing polymerase chain reaction (PCR) on the extracted DNA using a pair of primers having SEQ ID NO:1 and SEQ ID NO: 2; and

(c) comparing an increase or decrease in content of bacteria-derived extracellular vesicles of the subject sample with that of a normal individual-derived sample, a chronic obstructive pulmonary disease patient-derived sample, or an asthma patient-derived sample through sequencing of a product of the PCR.

The present invention also provides a method of predicting a risk for lung cancer, comprising the following processes:

(a) extracting DNA from extracellular vesicles isolated from a subject sample;

(b) performing polymerase chain reaction (PCR) on the extracted DNA using a pair of primers having SEQ ID NO:1 and SEQ ID NO: 2; and

(c) comparing an increase or decrease in content of bacteria-derived extracellular vesicles of the subject sample with that of a normal individual-derived sample, a chronic obstructive pulmonary disease patient-derived sample, or an asthma patient-derived sample through sequencing of a product of the PCR.

In one embodiment of the present invention, in process (c), lung cancer may be diagnosed by comparing an increase or decrease in content of extracellular vesicles derived from one or more bacteria selected from the group consisting of the phylum Acidobacteria and the phylum Chloroflexi with that of the normal individual-derived sample.

In another embodiment of the present invention, in process (c), lung cancer may be diagnosed by comparing an increase or decrease in content of extracellular vesicles derived from one or more bacteria selected from the group consisting of the class Thermomicrobia and the class Solibacteres with that of the normal individual-derived sample.

In another embodiment of the present invention, in process (c), lung cancer may be diagnosed by comparing an increase or decrease in content of extracellular vesicles derived from one or more bacteria selected from the group consisting of the order Turicibacterales, the order Rickettsiales, the order Alteromonadales, the order RF32, the order Thermales, the order JG30-KF-CM45, the order 1025, the order Solibacterales, and the order Aeromonadales with that of the normal individual-derived sample.

In another embodiment of the present invention, in process (c), lung cancer may be diagnosed by comparing an increase or decrease in content of extracellular vesicles derived from one or more bacteria selected from the group consisting of the family Turicibacteraceae, the family Clostridiaceae, the family S24-7, the family Rhizobiaceae, the family mitochondria, the family F16, the family Gordoniaceae, the family Rhodospirillaceae, the family Thermaceae, the family Shewanellaceae, the family Ellin6075, the family Rs-045, and the family Aeromonadaceae with that of the normal individual-derived sample.

In another embodiment of the present invention, in process (c), lung cancer may be diagnosed by comparing an increase or decrease in content of extracellular vesicles derived from one or more bacteria selected from the group consisting of the genus Chromohalobacter, the genus Geobacillus, the genus Proteus, the genus Megamonas, the genus Moraxella, the genus Alloiococcus, the genus Turicibacter, the genus SMB53, the genus Veillonella, the genus Peptoniphilus, the genus Comamonas, the genus Hymenobacter, the genus Citrobacter, the genus Novosphingobium, the genus Gordonia, the genus Aerococcus, the genus Thermus, the genus Shewanella, and the genus Achromobacter with that of the normal individual-derived sample.

In another embodiment of the present invention, in process (c), lung cancer may be diagnosed by comparing an increase or decrease in content of extracellular vesicles derived from one or more bacteria selected from the group consisting of the order Bacillales, the order Rickettsiales, and the order I025 with that of the chronic obstructive pulmonary disease patient-derived sample.

In another embodiment of the present invention, in process (c), lung cancer may be diagnosed by comparing an increase or decrease in content of extracellular vesicles derived from one or more bacteria selected from the group consisting of the family Staphylococcaceae, the family Nocardiaceae, and the family Rs-045 with that of the chronic obstructive pulmonary disease patient-derived sample.

In another embodiment of the present invention, in process (c), lung cancer may be diagnosed by comparing an increase or decrease in content of extracellular vesicles derived from one or more bacteria selected from the group consisting of the genus Alloiococcus, the genus Moraxella, the genus Staphylococcus, the genus Brevundimonas, the genus Enhydrobacter, the genus Comamonas, and the genus Rhodococcus with that of the chronic obstructive pulmonary disease patient-derived sample.

In another embodiment of the present invention, in process (c), lung cancer may be diagnosed by comparing an increase or decrease in content of extracellular vesicles derived from one or more bacteria selected from the group consisting of the phylum Bacteroidetes, the phylum Cyanobacteria, the phylum TM7, the phylum Fusobacteria, the phylum Thermi, the phylum Verrucomicrobia, the phylum Armatimonadetes, the phylum Acidobacteria, the phylum Gemmatimonadetes, and the phylum Chloroflexi with that of the asthma patient-derived sample.

In another embodiment of the present invention, in process (c), lung cancer may be diagnosed by comparing an increase or decrease in content of extracellular vesicles derived from one or more bacteria selected from the group consisting of the class Bacteroidia, the class Bacilli, the class Flavobacteriia, the class Sphingobacteriia, the class Alphaproteobacteria, the class Fusobacteriia, the class TM7-3, the class Deinococci, the class Verrucomicrobiae, the class Saprospirae, the class Chloroplast, the class Cytophagia, the class Fimbriimonadia, the class Chloracidobacteria, the class Thermomicrobia, the class Thermoleophilia, and the class Solibacteres with that of the asthma patient-derived sample.

In another embodiment of the present invention, in process (c), lung cancer may be diagnosed by comparing an increase or decrease in content of extracellular vesicles derived from one or more bacteria selected from the group consisting of the order YS2, the order Turicibacterales, the order Bifidobacteriales, the order Bacteroidales, the order Enterobacteriales, the order Rhodobacterales, the order Gemellales, the order Flavobacteriales, the order Caulobacterales, the order Neisseriales, the order Sphingobacteriales, the order Deinococcales, the order Pseudomonadales, the order Rhodocyclales, the order Xanthomonadales, the order Fusobacteriales, the order Actinomycetales, the order Sphingomonadales, the order Verrucomicroblates, the order Saprospirales, the order Rhizobiales, the order Bacillales, the order Streptophyta, the order Cytophagales, the order Thermales, the order Fimbriimonadales, the order CW040, the order Rickettsiales, the order RB41, the order Alteromonadales, the order JG30-KF-CM45, the order 1025, the order Aeromonadales, and the order Solibacterales with that of the asthma patient-derived sample.

In another embodiment of the present invention, in process (c), lung cancer may be diagnosed by comparing an increase or decrease in content of extracellular vesicles derived from one or more bacteria selected from the group consisting of the family Helicobacteraceae, the family Bacteroidaceae, the family Turicibacteraceae, the family Veillonellaceae, the family Bifidobacteriaceae, the family Barnesiellaceae, the family Rikenellaceae, the family Clostridiaceae, the family Odoribacteraceae, the family Enterobacteriaceae, the family Porphyromonadaceae, the family Gemellaceae, the family Weeksellaceae, the family Carnobacteriaceae, the family Leptotrichiaceae, the family Moraxellaceae, the family Caulobacteraceae, the family Erythrobacteraceae, the family Hyphomicrobiaceae, the family Neisseriaceae, the family Sphingobacteriaceae, the family Deinococcaceae, the family Aerococcaceae, the family Bartonellaceae, the family Micrococcaceae, the family Flavobacteriaceae, the family Burkholderiaceae, the family Lactobacillaceae, the family Dietziaceae, the family Rhodocyclaceae, the family Xanthomonadaceae, the family Geodermatophilaceae, the family Actinomycetaceae, the family Methylobacteriaceae, the family Pseudomonadaceae, the family Corynebacteriaceae, the family Staphylococcaceae, the family Nocardioidaceae, the family Verrucomicrobiaceae, the family Sphingomonadaceae, the family Mycobacteriaceae, the family Tissierellaceae, the family Chitinophagaceae, the family Intrasporangiaceae, the family Propionibacteriaceae, the family Aurantimonadaceae, the family Planococcaceae, the family Fusobacteriaceae, the family Bradyrhizobiaceae, the family Nocardiaceae, the family Dermabacteraceae, the family Bacillaceae, the family Thermaceae, the family Ellin6075, the family Brevibacteriaceae, the family Microbacteriaceae, the family Rhodospirillaceae, the family Cytophagaceae, the family Fimbriimonadaceae, the family Dermacoccaceae, the family Chromatiaceae, the family Rhizobiaceae, the family Gordoniaceae, the family mitochondria, the family Pseudonocardiaceae, the family Exiguobacteraceae, the family Shewanellaceae, the family F16, the family Rs-045, and the family Aeromonadaceae with that of the asthma patient-derived sample.

In another embodiment of the present invention, in process (c), lung cancer may be diagnosed by comparing an increase or decrease in content of extracellular vesicles derived from one or more bacteria selected from the group consisting of the genus Trabulsiella, the genus Enterobacter, the genus Veillonella, the genus Bifidobacterium, the genus Lachnospira, the genus Comamonas, the genus Bacteroides, the genus Turicibacter, the genus Sutterella, the genus Klebsiella, the genus SMB53, the genus Roseburia, the genus Dialister, the genus Ruminococcus, the genus Parabacteroides, the genus Butyricimonas, the genus Odoribacter, the genus Eubacterium, the genus Dorea, the genus Enhydrobacter, the genus Granulicatella, the genus Chryseobacterium, the genus Porphyromonas, the genus Coprococcus, the genus Peptoniphilus, the genus Microbispora, the genus Deinococcus, the genus Acinetobacter, the genus Aerococcus, the genus Actinomyces, the genus Brevundimonas, the genus Blastomonas, the genus Citrobacter, the genus Lactobacillus, the genus Stenotrophomonas, the genus Corynebacterium, the genus Pseudomonas, the genus Lautropia, the genus Akkermansia, the genus Staphylococcus, the genus Bacillus, the genus Sphingobacterium, the genus Anaerococcus, the genus Neisseria, the genus Leptotrichia, the genus Mycobacterium, the genus Kocuria, the genus Methylobacterium, the genus Propionibacterium, the genus Hymenobacter, the genus Sphingomonas, the genus Fusobacterium, the genus Brachybacterium, the genus Rhodococcus, the genus Micrococcus, the genus Kaistobacter, the genus Finegoldia, the genus Rubellimicrobium, the genus Brevibacterium, the genus Agrobacterium, the genus Dietzia, the genus Fimbriimonas, the genus Flavobacterium, the genus Dermacoccus, the genus Skermanella, the genus Novosphingobium, the genus Gordonia, the genus Rheinheimera, the genus Achromobacter, the genus Hydrogenophilus, the genus Therms, the genus Exiguobacterium, the genus Shewanella, the genus Ralstonia, and the genus Alkanindiges with that of the asthma patient-derived sample.

In another embodiment of the present invention, the subject sample may be blood.

In another embodiment of the present invention, the blood may be whole blood, serum, plasma, or blood mononuclear cells.

### Advantageous Effects

Extracellular vesicles secreted from bacteria present in the environment are absorbed into the human body, and thus can directly affect the occurrence of cancer, and since it is difficult to implement early diagnosis for lung cancer before symptoms are shown, efficient treatment is difficult. Thus, according to the present invention, a risk for lung cancer may be predicted through metagenomic analysis of bacteria or bacteria-derived extracellular vesicles using a human body-derived sample, and thus the onset of lung cancer can be delayed or lung cancer can be prevented through appropriate management by early diagnosis and prediction of a risk group for lung cancer, and, even after lung cancer occurs, early diagnosis for lung cancer can be implemented, thereby lowering a disease rate and increasing therapeutic effects. In addition, causative factors can be predicted by performing metagenomic analysis on patients diagnosed with lung cancer, and thus the patients are able to avoid exposure to the causative factors, whereby the progression of lung cancer is ameliorated, or recurrence of lung cancer can be prevented.

### DESCRIPTION OF DRAWINGS

FIGS. 1A and 1B are views for evaluating the distribution pattern of extracellular vesicles derived from bacteria in vivo. FIG. 1A illustrates images showing the distribution pattern of intestinal bacteria and extracellular vehicles (EVs) derived from bacteria per time (0 h, 5 min, 3 h, 6 h, and 12 h) after being orally administered to mice. FIG. 1B illustrates images showing the distribution pattern of intestinal bacteria and EVs derived from bacteria after being orally administered to mice and, after 12 hours, blood and various organs (heart, lung, liver, kidney, spleen, adipose tissue, and muscle) of the mice were extracted.

FIG. 2 illustrates distribution results of bacteria-derived EVs exhibiting significant diagnostic performance at a phylum level, after metagenomic analysis of bacteria-derived EVs isolated from lung cancer patient-derived blood and normal individual-derived blood.

FIG. 3 illustrates distribution results of bacteria-derived EVs exhibiting significant diagnostic performance at a class level, after metagenomic analysis of bacteria-derived EVs isolated from lung cancer patient-derived blood and normal individual-derived blood.

FIG. 4 illustrates distribution results of bacteria-derived EVs exhibiting significant diagnostic performance at an order level, after metagenomic analysis of bacteria-derived EVs isolated from lung cancer patient-derived blood and normal individual-derived blood.

FIG. 5 illustrates distribution results of bacteria-derived EVs exhibiting significant diagnostic performance at a family level, after metagenomic analysis of bacteria-derived EVs isolated from lung cancer patient-derived blood and normal individual-derived blood.

FIG. 6 illustrates distribution results of bacteria-derived EVs exhibiting significant diagnostic performance at a genus level, after metagenomic analysis of bacteria-derived EVs isolated from lung cancer patient-derived blood and normal individual-derived blood.

FIG. 7 illustrates distribution results of bacteria-derived EVs exhibiting a significant diagnostic performance at an order level, after metagenomic analysis of bacteria-derived EVs isolated from lung cancer patient-derived blood and COPD patient-derived blood.

FIG. 8 illustrates distribution results of bacteria-derived EVs exhibiting significant diagnostic performance at a family level, after metagenomic analysis of bacteria-derived EVs isolated from lung cancer patient-derived blood and COPD patient-derived blood.

FIG. 9 illustrates distribution results of bacteria-derived EVs exhibiting significant diagnostic performance at a genus level, after metagenomic analysis of bacteria-derived EVs isolated from lung cancer patient-derived blood and COPD patient-derived blood.

FIG. 10 illustrates distribution results of bacteria-derived EVs exhibiting significant diagnostic performance at a phylum level, after metagenomic analysis of bacteria-derived EVs isolated from lung cancer patient-derived blood and asthma patient-derived blood.

FIG. 11 illustrates distribution results of bacteria-derived EVs exhibiting significant diagnostic performance at a class level, after metagenomic analysis of bacteria-derived EVs isolated from lung cancer patient-derived blood and asthma patient-derived blood.

FIG. 12 illustrates distribution results of bacteria-derived EVs exhibiting significant diagnostic performance at an order level, after metagenomic analysis of bacteria-derived EVs isolated from lung cancer patient-derived blood and asthma patient-derived blood.

FIG. 13 illustrates distribution results of bacteria-derived EVs exhibiting significant diagnostic performance at a family level, after metagenomic analysis of bacteria-derived EVs isolated from lung cancer patient-derived blood and asthma patient-derived blood.

FIG. 14 illustrates distribution results of bacteria-derived EVs exhibiting significant diagnostic performance at a genus level, after metagenomic analysis of bacteria-derived EVs isolated from lung cancer patient-derived blood and asthma patient-derived blood.

### MODE OF THE INVENTION

The present invention relates to a method of diagnosing lung cancer through bacterial metagenomic analysis. The inventors of the present invention extracted genes from bacteria-derived extracellular vesicles of a subject-derived sample, performed metagenomic analysis thereon, and identified bacteria-derived extracellular vesicles capable of acting as a causative factor of lung cancer.

Thus, the present invention provides a method of providing information for lung cancer diagnosis, the method comprising:

(a) extracting DNA from extracellular vesicles isolated from a subject sample;

(b) performing polymerase chain reaction (PCR) on the extracted DNA using a pair of primers having SEQ ID NOS: 1 and 2; and

(c) comparing an increase or decrease in content of bacteria-derived extracellular vesicles of the subject sample with that of a normal individual-derived sample, a chronic obstructive pulmonary disease patient-derived sample, or an asthma patient-derived sample through sequencing of a product of the PCR.

The term “lung cancer diagnosis” as used herein refers to determining whether a patient has a risk for lung cancer, whether the risk for lung cancer is relatively high, or whether lung cancer has already occurred. The method of the present invention may be used to delay the onset of lung cancer through special and appropriate care for a specific patient, which is a patient having a high risk for lung cancer or prevent the onset of lung cancer. In addition, the method may be clinically used to determine treatment by selecting the most appropriate treatment method through early diagnosis of lung cancer.

The lung cancer diagnosis of the present invention is advantageous in that various subtypes of lung cancer, such as adenocarcinoma, small cell carcinoma, and squamous cell carcinoma can be simultaneously predicted.

The term “metagenome” as used herein refers to the total of genomes including all viruses, bacteria, fungi, and the like in isolated regions such as soil, the intestines of animals, and the like, and is mainly used as a concept of genomes that explains identification of many microorganisms at once using a sequencer to analyze non-cultured microorganisms. In particular, a metagenome does not refer to a genome of one species, but refers to a mixture of genomes, including genomes of all species of an environmental unit. This term originates from the view that, when defining one species in a process in which biology is advanced into omics, various species as well as existing one species functionally interact with each other to form a complete species. Technically, it is the subject of techniques that analyzes all DNAs and RNAs regardless of species using rapid sequencing to identify all species in one environment and verify interactions and metabolism. In the present invention, metagenomic analysis is performed using bacteria-derived extracellular vesicles isolated from serum.

In the present invention, the subject sample may be blood, and the blood may be whole blood, serum, plasma, or blood mononuclear cells, but the present invention is not limited thereto.

In an embodiment of the present invention, metagenomic analysis was performed on bacteria-derived extracellular vesicles in normal individual-derived blood and lung cancer patient-derived blood, and bacteria-derived extracellular vesicles capable of acting as a cause of the onset of lung cancer were actually identified by analysis at phylum, class, order, family, and genus levels.

More particularly, in one embodiment of the present invention, as a result of performing metagenomic analysis on bacteria-derived extracellular vesicles at a phylum level, the content of extracellular vesicles derived from bacteria belonging to the phylum Acidobacteria and the phylum Chloroflexi was significantly different between lung cancer patients and normal individuals (see Example 4).

More particularly, in one embodiment of the present invention, as a result of performing metagenomic analysis on bacteria-derived extracellular vesicles at a class level, the content of extracellular vesicles derived from bacteria belonging to the class Thermomicrobia and the class Solibacteres was significantly different between lung cancer patients and normal individuals (see Example 4).

More particularly, in one embodiment of the present invention, as a result of performing metagenomic analysis on bacteria-derived extracellular vesicles at an order level, the content of extracellular vesicles derived from bacteria belonging to the order Turicibacterales, the order Rickettsiales, the order Alteromonadales, the order RF32, the order Thermales, the order JG30-KF-CM45, the order 1025, the order Solibacterales, and the order Aeromonadales was significantly different between lung cancer patients and normal individuals (see Example 4).

More particularly, in one embodiment of the present invention, as a result of performing metagenomic analysis on bacteria-derived extracellular vesicles at a family level, the content of extracellular vesicles derived from bacteria belonging to the family Turicibacteraceae, the family Clostridiaceae, the family S24-7, the family Rhizobiaceae, the family mitochondria, the family F16, the family Gordoniaceae, the family Rhodospirillaceae, the family Thermaceae, the family Shewanellaceae, the family Ellin6075, the family Rs-045, and the family Aeromonadaceae was significantly different between lung cancer patients and normal individuals (see Example 4).

More particularly, in one embodiment of the present invention, as a result of performing metagenomic analysis on bacteria-derived extracellular vesicles at a genus level, the content of extracellular vesicles derived from bacteria belonging to the genus Chromohalobacter, the genus Geobacillus, the genus Proteus, the genus Megamonas, the genus Moraxella, the genus Alloiococcus, the genus Turicibacter, the genus SMB53, the genus Veillonella, the genus Peptoniphilus, the genus Comamonas, the genus Hymenobacter, the genus Citrobacter, the genus Novosphingobium, the genus Gordonia, the genus Aerococcus, the genus Thermus, the genus Shewanella, and the genus Achromobacter was significantly different between lung cancer patients and normal individuals (see Example 4).

In another embodiment of the present invention, metagenomic analysis was performed on bacteria-derived extracellular vesicles in chronic obstructive pulmonary disease (COPD) patient-derived blood and lung cancer patient-derived blood, and bacteria-derived extracellular vesicles capable of acting as a cause of the onset of lung cancer were actually identified by analysis at phylum, class, order, family, and genus levels.

More particularly, in one embodiment of the present invention, as a result of performing metagenomic analysis on bacteria-derived extracellular vesicles at an order level, the content of extracellular vesicles derived from bacteria belonging to the order Bacillales, the order Rickettsiales, and the order I025 was significantly different between lung cancer patients and COPD patients (see Example 5).

More particularly, in one embodiment of the present invention, as a result of performing metagenomic analysis on bacteria-derived extracellular vesicles at a family level, the content of extracellular vesicles derived from bacteria belonging to the family Staphylococcaceae, the family Nocardiaceae, and the family Rs-045 was significantly different between lung cancer patients and COPD patients (see Example 5).

More particularly, in one embodiment of the present invention, as a result of performing metagenomic analysis on bacteria-derived extracellular vesicles at a genus level, the content of extracellular vesicles derived from bacteria belonging to the genus Alloiococcus, the genus Moraxella, the genus Staphylococcus, the genus Brevundimonas, the genus Enhydrobacter, the genus Comamonas, and the genus Rhodococcus was significantly different between lung cancer patients and COPD patients (see Example 5).

In another embodiment of the present invention, metagenomic analysis was performed on bacteria-derived extracellular vesicles in asthma patient-derived blood and lung cancer patient-derived blood, and bacteria-derived extracellular vesicles capable of acting as a cause of the onset of lung cancer were actually identified by analysis at phylum, class, order, family, and genus levels.

More particularly, in one embodiment of the present invention, as a result of performing metagenomic analysis on bacteria-derived extracellular vesicles at a phylum level, the content of extracellular vesicles derived from bacteria belonging to the phylum Bacteroidetes, the phylum Cyanobacteria, the phylum TM7, the phylum Fusobacteria, the phylum Thermi, the phylum Verrucomicrobia, the phylum Armatimonadetes, the phylum Acidobacteria, the phylum Gemmatimonadetes, and the phylum Chloroflexi was significantly different between lung cancer patients and asthma patients (see Example 6).

More particularly, in one embodiment of the present invention, as a result of performing metagenomic analysis on bacteria-derived extracellular vesicles at a class level, the content of extracellular vesicles derived from bacteria belonging to the class Bacteroidia, the class Bacilli, the class Flavobacteriia, the class Sphingobacteriia, the class Alphaproteobacteria, the class Fusobacteriia, the class TM7-3, the class Deinococci, the class Verrucomicrobiae, the class Saprospirae, the class Chloroplast, the class Cytophagia, the class Fimbriimonadia, the class Chloracidobacteria, the class Thermomicrobia, the class Thermoleophilia, and the class Solibacteres was significantly different between lung cancer patients and asthma patients (see Example 6).

More particularly, in one embodiment of the present invention, as a result of performing metagenomic analysis on bacteria-derived extracellular vesicles at an order level, the content of extracellular vesicles derived from bacteria belonging to the order YS2, the order Turicibacterales, the order Bifidobacteriales, the order Bacteroidales, the order Enterobacteriales, the order Rhodobacterales, the order Gemellales, the order Flavobacteriales, the order Caulobacterales, the order Neisseriales, the order Sphingobacteriales, the order Deinococcales, the order Pseudomonadales, the order Rhodocyclales, the order Xanthomonadales, the order Fusobacteriales, the order Actinomycetales, the order Sphingomonadales, the order Verrucomicrobiales, the order Saprospirales, the order Rhizobiales, the order Bacillales, the order Streptophyta, the order Cytophagales, the order Thermales, the order Fimbriimonadales, the order CW040, the order Rickettsiales, the order RB41, the order Alteromonadales, the order JG30-KF-CM45, the order I025, the order Aeromonadales, and the order Solibacterales was significantly different between lung cancer patients and asthma patients (see Example 6).

More particularly, in one embodiment of the present invention, as a result of performing metagenomic analysis on bacteria-derived extracellular vesicles at a family level, the content of extracellular vesicles derived from bacteria belonging to the family Helicobacteraceae, the family Bacteroidaceae, the family Turicibacteraceae, the family Veillonellaceae, the family Bifidobacteriaceae, the family Barnesiellaceae, the family Rikenellaceae, the family Clostridiaceae, the family Odoribacteraceae, the family Enterobacteriaceae, the family Porphyromonadaceae, the family Gemellaceae, the family Weeksellaceae, the family Carnobacteriaceae, the family Leptotrichiaceae, the family Moraxellaceae, the family Caulobacteraceae, the family Erythrobacteraceae, the family Hyphomicrobiaceae, the family Neisseriaceae, the family Sphingobacteriaceae, the family Deinococcaceae, the family Aerococcaceae, the family Bartonellaceae, the family Micrococcaceae, the family Flavobacteriaceae, the family Burkholderiaceae, the family Lactobacillaceae, the family Dietziaceae, the family Rhodocyclaceae, the family Xanthomonadaceae, the family Geodermatophilaceae, the family Actinomycetaceae, the family Methylobacteriaceae, the family Pseudomonadaceae, the family Corynebacteriaceae, the family Staphylococcaceae, the family Nocardioidaceae, the family Verrucomicrobiaceae, the family Sphingomonadaceae, the family Mycobacteriaceae, the family Tissierellaceae, the family Chitinophagaceae, the family Intrasporangiaceae, the family Propionibacteriaceae, the family Aurantimonadaceae, the family Planococcaceae, the family Fusobacteriaceae, the family Bradyrhizobiaceae, the family Nocardiaceae, the family Dermabacteraceae, the family Bacillaceae, the family Thermaceae, the family Ellin6075, the family Brevibacteriaceae, the family Microbacteriaceae, the family Rhodospirillaceae, the family Cytophagaceae, the family Fimbriimonadaceae, the family Dermacoccaceae, the family Chromatiaceae, the family Rhizobiaceae, the family Gordoniaceae, the family mitochondria, the family Pseudonocardiaceae, the family Exiguobacteraceae, the family Shewanellaceae, the family F16, the family Rs-045, and the family Aeromonadaceae was significantly different between lung cancer patients and asthma patients (see Example 6).

More particularly, in one embodiment of the present invention, as a result of performing metagenomic analysis on bacteria-derived extracellular vesicles at a genus level, the content of extracellular vesicles derived from bacteria belonging to the genus Trabulsiella, the genus Enterobacter, the genus Veillonella, the genus Bifidobacterium, the genus Lachnospira, the genus Comamonas, the genus Bacteroides, the genus Turicibacter, the genus Sutterella, the genus Klebsiella, the genus SMB53, the genus Roseburia, the genus Dialister, the genus Ruminococcus, the genus Parabacteroides, the genus Butyricimonas, the genus Odoribacter, the genus Eubacterium, the genus Dorea, the genus Enhydrobacter, the genus Granulicatella, the genus Chryseobacterium, the genus Porphyromonas, the genus Coprococcus, the genus Peptomphilus, the genus Microbispora, the genus Deinococcus, the genus Acinetobacter, the genus Aerococcus, the genus Actinomyces, the genus Brevundimonas, the genus Blastomonas, the genus Citrobacter, the genus Lactobacillus, the genus Stenotrophomonas, the genus Corynebacterium, the genus Pseudomonas, the genus Lautropia, the genus Akkermansia, the genus Staphylococcus, the genus Bacillus, the genus Sphingobacterium, the genus Anaerococcus, the genus Neisseria, the genus Leptotrichia, the genus Mycobacterium, the genus Kocuria, the genus Methylobacterium, the genus Propionibacterium, the genus Hymenobacter, the genus Sphingomonas, the genus Fusobacterium, the genus Brachybacterium, the genus Rhodococcus, the genus Micrococcus, the genus Kaistobacter, the genus Finegoldia, the genus Rubellimicrobium, the genus Brevibacterium, the genus Agrobacterium, the genus Dietzia, the genus Fimbriimonas, the genus Flavobacterium, the genus Dermacoccus, the genus Skermanella, the genus Novosphingobium, the genus Gordonia, the genus Rheinheimera, the genus Achromobacter, the genus Hydrogenophilus, the genus Thermus, the genus Exiguobacterium, the genus Shewanella, the genus Ralstonia, and the genus Alkanindiges was significantly different between lung cancer patients and asthma patients (see Example 6).

From the above-described example results, it was confirmed that bacteria-derived extracellular vesicles exhibiting a significant change in content in lung cancer patients compared to normal individuals, COPD patients, and asthma patients, are identified by performing metagenomic analysis on bacteria-derived extracellular vesicles isolated from blood, and lung cancer may be diagnosed by analyzing an increase or decrease in the content of bacteria-derived extracellular vesicles at each level through metagenomic analysis.

Hereinafter, the present invention will be described with reference to exemplary examples to aid in understanding of the present invention. However, these examples are provided only for illustrative purposes and are not intended to limit the scope of the present invention.

### EXAMPLES

### Example 1

### Analysis of In Vivo Absorption, Distribution, and Excretion Patterns of Intestinal Bacteria and Bacteria-Derived Extracellular Vesicles

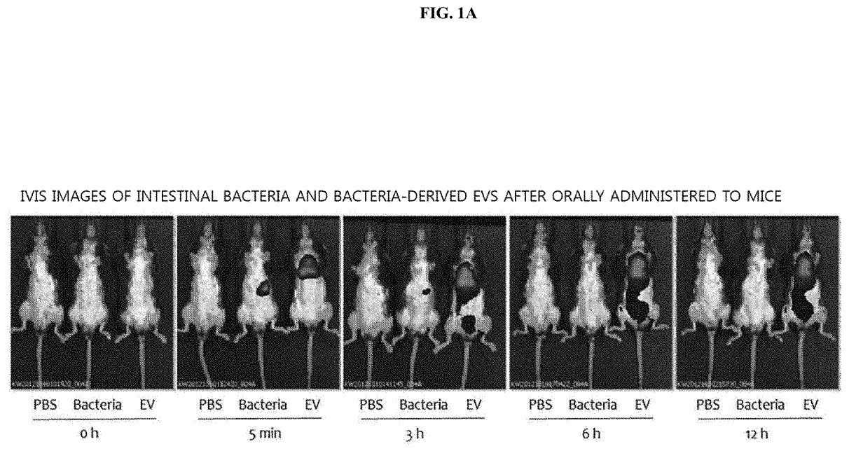

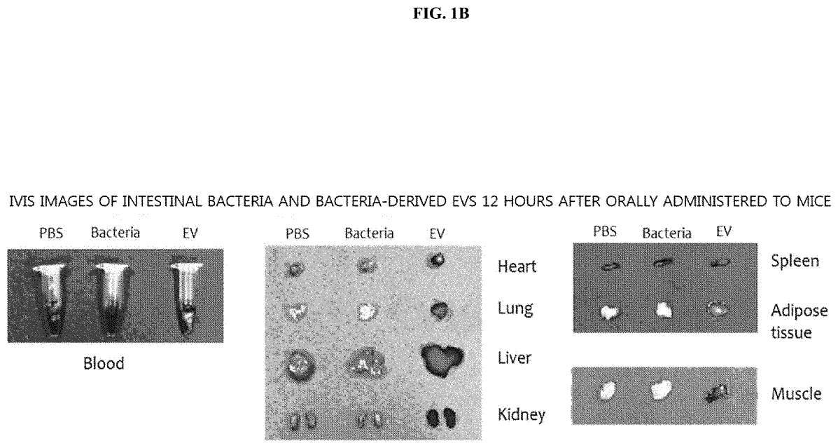

To evaluate whether intestinal bacteria and bacteria-derived extracellular vesicles are systematically absorbed through the gastrointestinal tract, an experiment was conducted using the following method. More particularly, 50 μg of each of intestinal bacteria and the bacteria-derived extracellular vesicles (EVs), labeled with fluorescence, were orally administered to the gastrointestinal tracts of mice, and fluorescence was measured at 0 h, and after 5 min, 3 h, 6 h, and 12 h. As a result of observing the entire images of mice, as illustrated in FIG. 1A, the bacteria were not systematically absorbed when administered, while the bacteria-derived EVs were systematically absorbed at 5 min after administration, and, at 3 h after administration, fluorescence was strongly observed in the bladder, from which it was confirmed that the EVs were excreted via the urinary system, and were present in the bodies up to 12 h after administration.

After intestinal bacteria and intestinal bacteria-derived extracellular vesicles were systematically absorbed, to evaluate a pattern of invasion of intestinal bacteria and the bacteria-derived EVs into various organs in the human body after being systematically absorbed, 50 μg of each of the bacteria and bacteria-derived EVs, labeled with fluorescence, were administered using the same method as that used above, and then, at 12 h after administration, blood, the heart, the lungs, the liver, the kidneys, the spleen, adipose tissue, and muscle were extracted from each mouse. As a result of observing fluorescence in the extracted tissues, as illustrated in FIG. 1B, it was confirmed that the intestinal bacteria were not absorbed into each organ, while the bacteria-derived EVs were distributed in the blood, heart, lungs, liver, kidneys, spleen, adipose tissue, and muscle.

### Example 2

### Vesicle Isolation and DNA Extraction from Blood

To isolate vesicles and extract DNA, from blood, first, blood was added to a 10 ml tube and centrifuged at 3,500×g and 4□ for 10 min to precipitate a suspension, and only a supernatant was collected, which was then placed in a new 10 ml tube. The collected supernatant was filtered using a 0.22 μm filter to remove bacteria and impurities, and then placed in centripreigugal filters (50 kD) and centrifuged at 1500×g and 4□ for 15 min to discard materials with a smaller size than 50 kD, and then concentrated to 10 ml. Once again, bacteria and impurities were removed therefrom using a 0.22 μm filter, and then the resulting concentrate was subjected to ultra-high speed centrifugation at 150,000×g and 4□ for 3 hours by using a Type 90ti rotor to remove a supernatant, and the agglomerated pellet was dissolved with phosphate-buffered saline (PBS), thereby obtaining vesicles.

100 μl of the vesicles isolated from the blood according to the above-described method was boiled at 100□ to allow the internal DNA to come out of the lipid and then cooled on ice for 5 minutes. Next, the resulting vesicles were centrifuged at 10,000×g and 4□ for 30 minutes to remove the remaining suspension, only the supernatant was collected, and then the amount of DNA extracted was quantified using a NanoDrop sprectrophotometer. In addition, to verify whether bacteria-derived DNA was present in the extracted DNA, PCR was performed using 16s rDNA primers shown in Table 1 below.

TABLE 1

| | | | SEQ |
|---|---|---|---|
| | | | ID |
| primer | | sequence | NO. |
| 16S | 16S_V3_F | 5′-TCGTCGGCAGCGTCAGATGTGTATAAG | 1 |
| rDNA | | AGACAGCCTACGGGNGGCWGCAG-3′ | |
| | 16S_V4_R | 5′-GTCTCGTGGGCTCGGAGATGTGTATAA | 2 |
| | | GAGACAGGACTACHVGGGTATCTAATCC-3′ | |

### Example 3

### Metagenomic Analysis Using DNA Extracted from Blood

DNA was extracted using the same method as that used in Example 2, and then PCR was performed thereon using 16S rDNA primers shown in Table 1 to amplify DNA, followed by sequencing (Illumina MiSeq sequencer). The results were output as standard flowgram format (SFF) files, and the SFF files were converted into sequence files (.fasta) and nucleotide quality score files using GS FLX software (v2.9), and then credit rating for reads was identified, and portions with a window (29 bps) average base call accuracy of less than 99% (Phred score<20) were removed. After removing the low-quality portions, only reads having a length of 300 bps or more were used (Sickle version 1.33), and, for operational taxonomy unit (OTU) analysis, clustering was performed using UCLUST and USEARCH according to sequence similarity. In particular, clustering was performed based on sequence similarity values of 94% for genus, 90% for family, 85% for order, 80% for class, and 75% for phylum, and phylum, class, order, family, and genus levels of each OTU were classified, and bacteria with a sequence similarity of 97% or more were analyzed (QIIME) using 16S DNA sequence databases (108,453 sequences) of BLASTN and GreenGenes.

### Example 4

### Lung Cancer Diagnostic Model Based on Metagenomic Analysis of Bacteria-Derived Extracellular Vesicles Isolated from Normal Individual-Derived Blood and Lung Cancer Patient-Derived Blood

Extracellular vesicles were isolated from blood samples of 318 lung cancer patients and 234 normal individuals, the two groups matched in age and gender, and then metagenomic sequencing was performed thereon using the method of Example 3. For the development of a diagnostic model, first, a strain exhibiting a p value of less than 0.05 between two groups in a t-test and a difference of two-fold or more between two groups was selected, and then an area under curve (AUC), sensitivity, and specificity, which are diagnostic performance indexes, were calculated by logistic regression analysis.

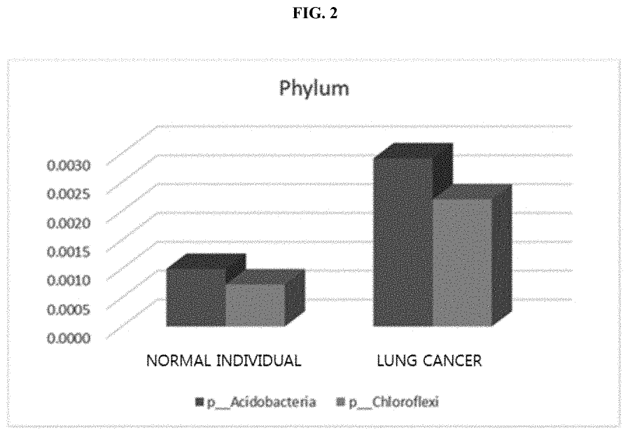

As a result of analyzing bacteria-derived extracellular vesicles in blood at a phylum level, a diagnostic model developed using, as a biomarker, one or more bacteria selected from the phylum Acidobacteria and the phylum Chloroflexi exhibited significant diagnostic performance for lung cancer (see Table 2 and FIG. 2).

TABLE 2

| | Normal Individual | | Lung Cancer | | t-test | | Training Set | | | Testing Set | | |
|---|---|---|---|---|---|---|---|---|---|---|---|---|
| Taxon | Mean | SD | Mean | SD | p-value | fold | AUC | sensitivity | specificity | AUC | sensitivity | specificity |
| p_Acidobacteria | 0.0010 | 0.0034 | 0.0029 | 0.0084 | 0.0002 | 2.92 | 0.73 | 0.79 | 0.53 | 0.71 | 0.76 | 0.55 |
| p_Chloroflexi | 0.0007 | 0.0030 | 0.0022 | 0.0047 | 0.0000 | 3.03 | 0.72 | 0.78 | 0.54 | 0.76 | 0.74 | 0.61 |

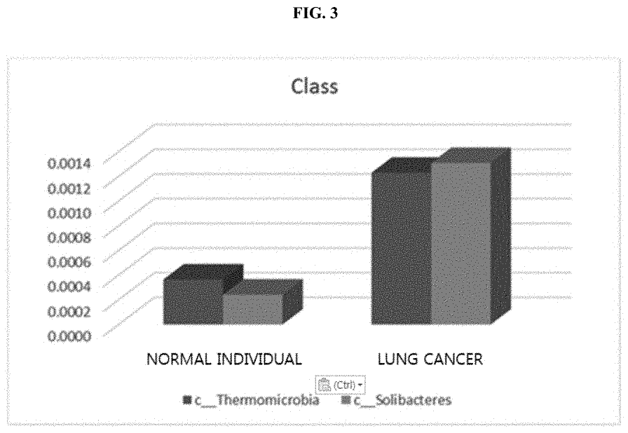

As a result of analyzing bacteria-derived extracellular vesicles in blood at a class level, a diagnostic model developed using, as a biomarker, one or more bacteria selected from the class Thermomicrobia and the class Solibacteres exhibited significant diagnostic performance for lung cancer (see Table 3 and FIG. 3).

TABLE 3

| | Normal Individual | | Lung Cancer | | t-test | | Training Set | | | Testing Set | | |
|---|---|---|---|---|---|---|---|---|---|---|---|---|
| Taxon | Mean | SD | Mean | SD | p-value | fold | AUC | sensitivity | specificity | AUC | sensitivity | specificity |
| c_Thermomicrobia | 0.0004 | 0.0014 | 0.0012 | 0.0033 | 0.0000 | 3.38 | 0.72 | 0.77 | 0.54 | 0.74 | 0.75 | 0.64 |
| c_Solibacteres | 0.0002 | 0.0018 | 0.0013 | 0.0051 | 0.0005 | 5.43 | 0.73 | 0.81 | 0.55 | 0.71 | 0.76 | 0.53 |

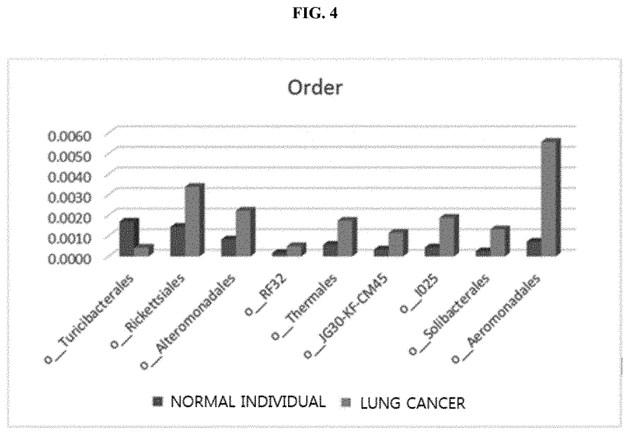

As a result of analyzing bacteria-derived extracellular vesicles in blood at an order level, a diagnostic model developed using, as a biomarker, one or more bacteria selected from the order Turicibacterales, the order Rickettsiales, the order Alteromonadales, the order RF32, the order Thermales, the order JG30-KF-CM45, the order 1025, the order Solibacterales, and the order Aeromonadales exhibited significant diagnostic performance for lung cancer (see Table 4 and FIG. 4).

TABLE 4

| | Normal Individual | | Lung Cancer | | t-test | | Training Set | | | Testing Set | | |
|---|---|---|---|---|---|---|---|---|---|---|---|---|
| Taxon | Mean | SD | Mean | SD | p-value | fold | AUC | sensitivity | specificity | AUC | sensitivity | specificity |
| o_Turicibacterales | 0.0017 | 0.0033 | 0.0004 | 0.0017 | 0.0000 | 0.25 | 0.76 | 0.75 | 0.63 | 0.74 | 0.69 | 0.70 |
| o_Rickettsiales | 0.0014 | 0.0053 | 0.0034 | 0.0089 | 0.0006 | 2.36 | 0.70 | 0.75 | 0.54 | 0.72 | 0.71 | 0.58 |
| o_Alteromonadales | 0.0008 | 0.0023 | 0.0022 | 0.0081 | 0.0032 | 2.71 | 0.70 | 0.76 | 0.56 | 0.72 | 0.69 | 0.59 |
| o_RF32 | 0.0007 | 0.0008 | 0.0005 | 0.0021 | 0.0082 | 3.07 | 0.70 | 0.77 | 0.56 | 0.72 | 0.70 | 0.57 |
| o_Thermales | 0.0006 | 0.0022 | 0.0017 | 0.0039 | 0.0000 | 3.10 | 0.71 | 0.78 | 0.56 | 0.75 | 0.76 | 0.59 |
| o_JG30-KF-CM45 | 0.0003 | 0.0014 | 0.0011 | 0.0033 | 0.0000 | 3.49 | 0.72 | 0.78 | 0.55 | 0.74 | 0.75 | 0.64 |
| o_I025 | 0.0004 | 0.0021 | 0.0019 | 0.0059 | 0.0000 | 4.36 | 0.73 | 0.81 | 0.56 | 0.72 | 0.73 | 0.55 |
| o_Solibaeterales | 0.0002 | 0.0018 | 0.0013 | 0.0051 | 0.0005 | 5.43 | 0.73 | 0.81 | 0.55 | 0.71 | 0.76 | 0.53 |
| o_Aeromonadales | 0.0007 | 0.0029 | 0.0056 | 0.0320 | 0.0077 | 7.88 | 0.71 | 0.79 | 0.55 | 0.72 | 0.72 | 0.55 |

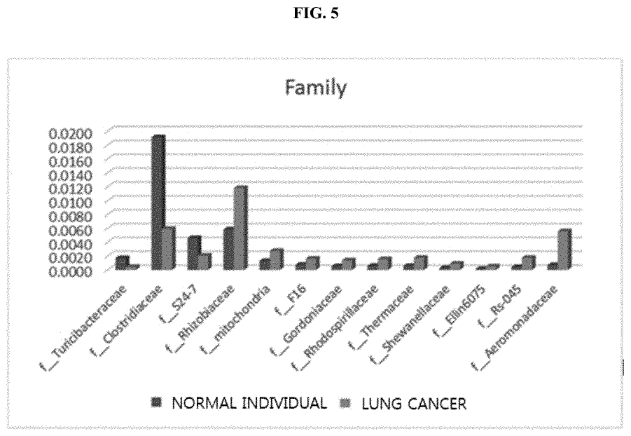

As a result of analyzing bacteria-derived extracellular vesicles in blood at a family level, a diagnostic model developed using, as a biomarker, one or more bacteria selected from the family Turicibacteraceae, the family Clostridiaceae, the family S24-7, the family Rhizobiaceae, the family mitochondria, the family F16, the family Gordoniaceae, the family Rhodospirillaceae, the family Thermaceae, the family Shewanellaceae, the family Ellin6075, the family Rs-045, and the family Aeromonadaceae exhibited significant diagnostic performance for lung cancer (see Table 5 and FIG. 5).

TABLE 5

| | Normal Individual | | Lung Cancer | | t-test | | Training Set | | | Testing Set | | |
|---|---|---|---|---|---|---|---|---|---|---|---|---|
| Taxon | Mean | SD | Mean | SD | p-value | fold | AUC | sensitivity | specificity | AUC | sensitivity | specificity |
| f_Turicibacteraceae | 0.0017 | 0.0033 | 0.0004 | 0.0017 | 0.0000 | 0.25 | 0.76 | 0.75 | 0.63 | 0.74 | 0.69 | 0.70 |
| f_Clostridiaceae | 0.0191 | 0.0450 | 0.0059 | 0.0078 | 0.0000 | 0.31 | 0.73 | 0.75 | 0.59 | 0.76 | 0.71 | 0.64 |
| f_S24-7 | 0.0046 | 0.0119 | 0.0020 | 0.0040 | 0.0000 | 0.44 | 0.69 | 0.75 | 0.55 | 0.71 | 0.69 | 0.59 |
| f_Rhizobiaceae | 0.0058 | 0.0082 | 0.0118 | 0.0150 | 0.0000 | 2.09 | 0.72 | 0.77 | 0.52 | 0.77 | 0.75 | 0.63 |
| f_mitochondria | 0.0013 | 0.0053 | 0.0027 | 0.0081 | 0.0065 | 2.12 | 0.69 | 0.75 | 0.56 | 0.72 | 0.69 | 0.59 |
| f_F16 | 0.0007 | 0.0032 | 0.0016 | 0.0040 | 0.0012 | 2.23 | 0.71 | 0.78 | 0.56 | 0.72 | 0.72 | 0.55 |
| f_Gordoniaceae | 0.0005 | 0.0024 | 0.0013 | 0.0048 | 0.0048 | 2.63 | 0.70 | 0.78 | 0.56 | 0.73 | 0.71 | 0.59 |
| f_Rhodospirillaceae | 0.0006 | 0.0022 | 0.0015 | 0.0062 | 0.0088 | 2.70 | 0.70 | 0.77 | 0.58 | 0.71 | 0.69 | 0.58 |
| f_Thermaceae | 0.0006 | 0.0022 | 0.0017 | 0.0039 | 0.0000 | 3.10 | 0.71 | 0.78 | 0.56 | 0.75 | 0.76 | 0.59 |
| f_Shewanellaceae | 0.0003 | 0.0012 | 0.0009 | 0.0040 | 0.0093 | 3.30 | 0.70 | 0.76 | 0.56 | 0.72 | 0.70 | 0.58 |
| f_Ellin6075 | 0.0001 | 0.0009 | 0.0005 | 0.0018 | 0.0020 | 3.73 | 0.71 | 0.77 | 0.56 | 0.73 | 0.71 | 0.57 |
| f_Rs-045 | 0.0004 | 0.0021 | 0.0017 | 0.0058 | 0.0001 | 4.22 | 0.72 | 0.81 | 0.54 | 0.72 | 0.73 | 0.55 |
| f_Aeromonadaceae | 0.0007 | 0.0029 | 0.0056 | 0.0320 | 0.0075 | 8.15 | 0.71 | 0.79 | 0.55 | 0.72 | 0.72 | 0.55 |

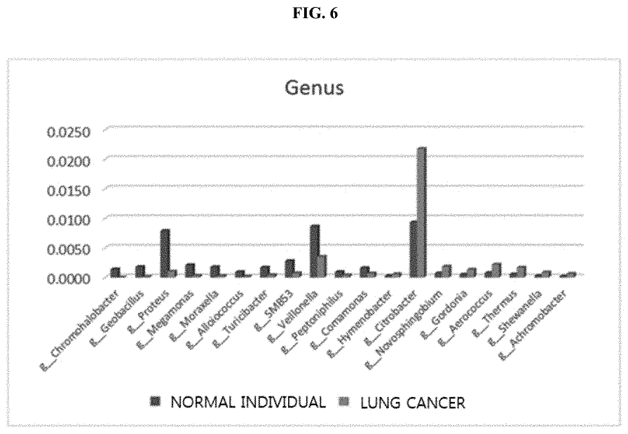

As a result of analyzing bacteria-derived extracellular vesicles in blood at a genus level, a diagnostic model developed using, as a biomarker, one or more bacteria selected from the genus Chromohalobacter, the genus Geobacillus, the genus Proteus, the genus Megamonas, the genus Moraxella, the genus Alloiococcus, the genus Turicibacter, the genus SMB53, the genus Veillonella, the genus Peptoniphilus, the genus Comamonas, the genus Hymenobacter, the genus Citrobacter, the genus Novosphingobium, the genus Gordonia, the genus Aerococcus, the genus Thermus, the genus Shewanella, and the genus Achromobacter exhibited significant diagnostic performance for lung cancer (see Table 6 and FIG. 6).

TABLE 6

| | Normal Individual | | Lung Cancer | | t-test | | Training Set | | | Testing Set | | |
|---|---|---|---|---|---|---|---|---|---|---|---|---|
| Taxon | Mean | SD | Mean | SD | p-value | fold | AUC | sensitivity | specificity | AUC | sensitivity | specificity |
| g_Chromohalobacter | 0.0014 | 0.0075 | 0.0000 | 0.0000 | 0.0001 | 0.00 | 0.72 | 0.72 | 0.63 | 0.75 | 0.69 | 0.69 |
| g_Geobacillus | 0.0018 | 0.0062 | 0.0001 | 0.0006 | 0.0000 | 0.06 | 0.70 | 0.74 | 0.59 | 0.71 | 0.66 | 0.66 |
| g_Proteus | 0.0079 | 0.0231 | 0.0010 | 0.0025 | 0.0000 | 0.13 | 0.76 | 0.76 | 0.62 | 0.75 | 0.69 | 0.69 |
| g_Megamonas | 0.0021 | 0.0097 | 0.0003 | 0.0014 | 0.0001 | 0.1.4 | 0.71 | 0.76 | 0.60 | 0.71 | 0.65 | 0.66 |
| g_Moraxella | 0.0018 | 0.0109 | 0.0003 | 0.0018 | 0.0055 | 0.15 | 0.70 | 0.76 | 0.57 | 0.71 | 0.65 | 0.63 |
| g_Alloiococcus | 0.0009 | 0.0041 | 0.0000 | 0.0008 | 0.0001 | 0.17 | 0.71 | 0.75 | 0.59 | 0.71 | 0.67 | 0.66 |
| g_Turicibactcr | 0.0017 | 0.0033 | 0.0004 | 0.0017 | 0.0000 | 0.25 | 0.76 | 0.75 | 0.63 | 0.74 | 0.69 | 0.70 |
| g_SMB53 | 0.0028 | 0.0078 | 0.0007 | 0.0015 | 0.0000 | 0.26 | 0.72 | 0.75 | 0.59 | 0.74 | 0.68 | 0.70 |
| g_Veillonella | 0.0086 | 0.0155 | 0.0035 | 0.0054 | 0.0000 | 0.41 | 0.71 | 0.74 | 0.59 | 0.74 | 0.70 | 0.65 |
| g_Peptoniphilus | 0.0009 | 0.0036 | 0.0004 | 0.0013 | 0.0034 | 0.41 | 0.70 | 0.76 | 0.56 | 0.71 | 0.66 | 0.63 |
| g_Comamonas | 0.0016 | 0.0038 | 0.0007 | 0.0023 | 0.0001 | 0.45 | 0.71 | 0.75 | 0.58 | 0.71 | 0.68 | 0.67 |
| g_Hymenobacter | 0.0003 | 0.0010 | 0.0006 | 0.0021 | 0.0095 | 2.26 | 0.70 | 0.76 | 0.56 | 0.71 | 0.69 | 0.57 |
| g_Citrobacter | 0.0093 | 0.0146 | 0.0218 | 0.0220 | 0.0000 | 2.33 | 0.74 | 0.81 | 0.54 | 0.81 | 0.85 | 0.61 |
| g_Novosphingobium | 0.0007 | 0.0027 | 0.0019 | 0.0043 | 0.0001 | 2.58 | 0.73 | 0.81 | 0.53 | 0.73 | 0.75 | 0.66 |
| g_Gordonia | 0.0005 | 0.0024 | 0.0013 | 0.0048 | 0.0047 | 2.64 | 0.70 | 0.78 | 0.56 | 0.73 | 0.71 | 0.59 |
| g_Aerococcus | 0.0008 | 0.0025 | 0.0022 | 0.0075 | 0.0013 | 2.86 | 0.71 | 0.79 | 0.54 | 0.70 | 0.72 | 0.55 |
| g_Thermus | 0.0006 | 0.0022 | 0.0017 | 0.0038 | 0.0000 | 3.02 | 0.71 | 0.78 | 0.56 | 0.74 | 0.76 | 0.58 |
| g_Shewanella | 0.0003 | 0.0012 | 0.0009 | 0.0040 | 0.0093 | 3.30 | 0.70 | 0.76 | 0.56 | 0.72 | 0.70 | 0.58 |
| g_Achromobacter | 0.0002 | 0.0011 | 0.0006 | 0.0028 | 0.0060 | 3.56 | 0.70 | 0.76 | 0.56 | 0.72 | 0.70 | 0.56 |

### Example 5

### Lung Cancer Diagnostic Model Based on Metagenomic Analysis of Bacteria-Derived Extracellular Vesicles Isolated from COPD Patient-Derived Blood and Lung Cancer Patient-Derived Blood

Extracellular vesicles were isolated from blood samples of 319 lung cancer patients and 208 COPD patients, and then metagenomic sequencing was performed thereon using the method of Example 3. For the development of a diagnostic model, first, a strain exhibiting a p value of less than 0.05 between two groups in a t-test and a difference of two-fold or more between two groups was selected, and then an AUC, sensitivity, and specificity, which are diagnostic performance indexes, were calculated by logistic regression analysis.

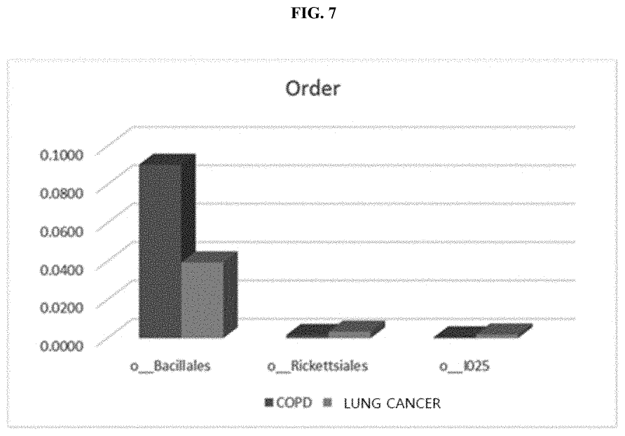

As a result of analyzing bacteria-derived extracellular vesicles in blood at an order level, a diagnostic model developed using, as a biomarker, one or more bacteria selected from the order Bacillales, the order Rickettsiales, and the order 1025 exhibited significant diagnostic performance for lung cancer (see Table 7 and FIG. 7).

TABLE 7

| | COPD | | Lung Cancer | | t-test | | Training Set | | | Testing Set | | |
|---|---|---|---|---|---|---|---|---|---|---|---|---|
| Taxon | Mean | SD | Mean | SD | p-value | fold | AUC | sensitivity | specificity | AUC | sensitivity | specificity |
| o_Bacillales | 0.0903 | 0.0740 | 0.0394 | 0.0509 | 0.0000 | 0.44 | 0.82 | 0.50 | 0.89 | 0.83 | 0.46 | 0.8 |
| o_Rickettsiales | 0.0015 | 0.0027 | 0.0034 | 0.0089 | 0.0007 | 2.18 | 0.58 | 0.00 | 0.99 | 0.60 | 0.00 | 1.00 |
| o_I025 | 0.0004 | 0.0012 | 0.0019 | 0.0059 | 0.0000 | 5.33 | 0.60 | 0.05 | 0.95 | 0.65 | 0.05 | 0.97 |

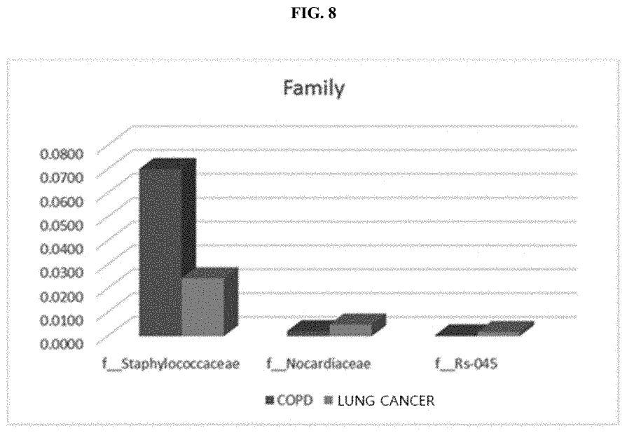

As a result of analyzing bacteria-derived extracellular vesicles in blood at a family level, a diagnostic model developed using, as a biomarker, one or more bacteria selected from the family Staphylococcaceae, the family Nocardiaceae, and the family Rs-045 exhibited significant diagnostic performance for lung cancer (see Table 8 and FIG. 8).

TABLE 8

| | COPD | | Lung Cancer | | t-test | | Training Set | | | Testing Set | | |
|---|---|---|---|---|---|---|---|---|---|---|---|---|
| Taxon | Mean | SD | Mean | SD | p-value | fold | AUC | sensitivity | specificity | AUC | sensitivity | specificity |
| f_Staphylococcaceae | 0.0700 | 0.0711 | 0.0242 | 0.0437 | 0.0000 | 0.35 | 0.85 | 0.53 | 0.94 | 0.84 | 0.52 | 0.86 |
| f_Nocardiaceae | 0.0020 | 0.0028 | 0.0048 | 0.0103 | 0.0000 | 2.35 | 0.61 | 0.19 | 0.80 | 0.61 | 0.18 | 0.83 |
| f_Rs-045 | 0.0003 | 0.0011 | 0.0017 | 0.0058 | 0.0000 | 5.49 | 0.60 | 0.06 | 0.95 | 0.64 | 0.07 | 0.95 |

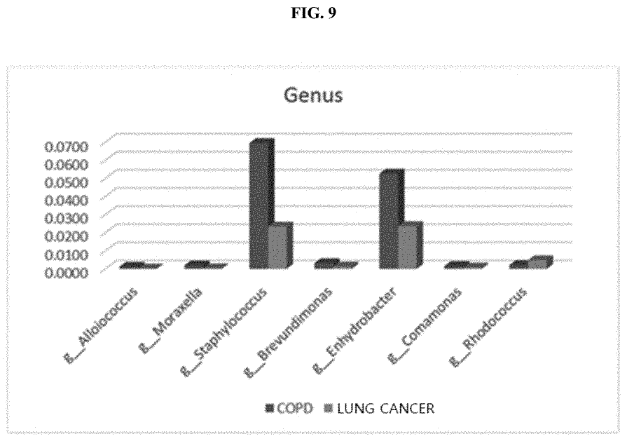

As a result of analyzing bacteria-derived extracellular vesicles in blood at a genus level, a diagnostic model developed using, as a biomarker, one or more bacteria selected from the genus Alloiococcus, the genus Moraxella, the genus Staphylococcus, the genus Brevundimonas, the genus Enhydrobacter, the genus Comamonas, and the genus Rhodococcus exhibited significant diagnostic performance for lung cancer (see Table 9 and FIG. 9).

TABLE 9

| | COPD | | Lung Cancer | | t-test | | Training Set | | | Testing Set | | |
|---|---|---|---|---|---|---|---|---|---|---|---|---|
| Taxon | Mean | SD | Mean | SD | p-value | fold | AUC | sensitivity | specificity | AUC | sensitivity | specificity |
| g_Alloiococcus | 0.0011 | 0.0037 | 0.0002 | 0.0008 | 0.0003 | 0.14 | 0.61 | 0.13 | 0.97 | 0.64 | 0.16 | 0.96 |
| g_Moraxella | 0.0019 | 0.0054 | 0.0003 | 0.0018 | 0.0001 | 0.15 | 0.64 | 0.14 | 0.98 | 0.63 | 0.13 | 0.98 |
| g_Staphylococcus | 0.0694 | 0.0711 | 0.0233 | 0.0435 | 0.0000 | 0.34 | 0.85 | 0.53 | 0.92 | 0.84 | 0.52 | 0.86 |
| g_Brevundimonas | 0.0031 | 0.0050 | 0.0012 | 0.0034 | 0.0000 | 0.40 | 0.66 | 0.19 | 0.93 | 0.71 | 0.20 | 0.95 |
| g_Enhydrobacter | 0.0525 | 0.0478 | 0.0236 | 0.0293 | 0.0000 | 0.45 | 0.77 | 0.35 | 0.92 | 0.79 | 0.39 | 0.88 |
| g_Comamonas | 0.0015 | 0.0026 | 0.0007 | 0.0023 | 0.0003 | 0.47 | 0.70 | 0.18 | 0.95 | 0.63 | 0.18 | 0.91 |
| g_Rhodococcus | 0.0020 | 0.0028 | 0.0047 | 0.0102 | 0.0000 | 2.31 | 0.61 | 0.19 | 0.80 | 0.61 | 0.18 | 0.83 |

### Example 6

### Lung Cancer Diagnostic Model Based on Metagenomic Analysis of Bacteria-Derived Extracellular Vesicles Isolated from Asthma Patient-Derived Blood and Lung Cancer Patient-Derived Blood

Extracellular vesicles were isolated from blood samples of 308 lung cancer patients and 277 asthma patients, and then metagenomic sequencing was performed thereon using the method of Example 3. For the development of a diagnostic model, first, a strain exhibiting a p value of less than 0.05 between two groups in a t-test and a difference of two-fold or more between two groups was selected, and then an AUC, sensitivity, and specificity, which are diagnostic performance indexes, were calculated by logistic regression analysis.

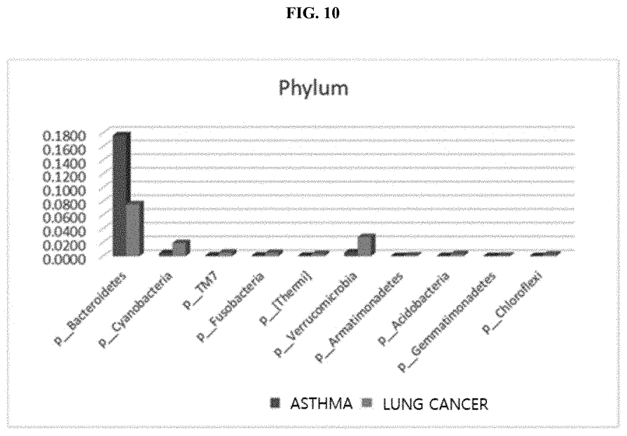

As a result of analyzing bacteria-derived extracellular vesicles in blood at a phylum level, a diagnostic model developed using, as a biomarker, one or more bacteria selected from the phylum Bacteroidetes, the phylum Cyanobacteria, the phylum TM7, the phylum Fusobacteria, the phylum Thermi, the phylum Verrucomicrobia, the phylum Armatimonadetes, the phylum Acidobacteria, the phylum Gemmatimonadetes, and the phylum Chloroflexi exhibited significant diagnostic performance for lung cancer (see Table 10 and FIG. 10).

TABLE 10

| | Asthma | | Lung Cancer | | t-test | | Training Set | | | Test Set | | |
|---|---|---|---|---|---|---|---|---|---|---|---|---|
| Taxon | Mean | SD | Mean | SD | p-value | fold | Auc | sensitivity | specificity | Auc | sensitivity | specificity |
| p_Bacteroidetes | 0.1762 | 0.0499 | 0.0757 | 0.0370 | 0.0000 | 0.43 | 0.97 | 0.88 | 0.93 | 0.96 | 0.90 | 0.91 |
| p_Cyanobacteria | 0.0043 | 0.0119 | 0.0189 | 0.0319 | 0.0000 | 4.39 | 0.89 | 0.70 | 0.86 | 0.85 | 0.75 | 0.80 |
| p_TM7 | 0.0010 | 0.0029 | 0.0051 | 0.0080 | 0.0000 | 4.98 | 0.87 | 0.65 | 0.90 | 0.78 | 0.69 | 0.74 |
| p_Fusobacteria | 0.0009 | 0.0029 | 0.0047 | 0.0095 | 0.0000 | 5.04 | 0.85 | 0.63 | 0.89 | 0.84 | 0.68 | 0.84 |
| p_[Thermi] | 0.0006 | 0.0019 | 0.0031 | 0.0065 | 0.0000 | 5.25 | 0.83 | 0.62 | 0.88 | 0.80 | 0.65 | 0.82 |
| p_Verrucomicrobia | 0.0053 | 0.0060 | 0.0277 | 0.0236 | 0.0000 | 5.25 | 0.91 | 0.79 | 0.82 | 0.87 | 0.78 | 0.77 |
| p_Armatimonadetes | 0.0001 | 0.0004 | 0.0009 | 0.0051 | 0.0046 | 8.82 | 0.81 | 0.62 | 0.87 | 0.76 | 0.65 | 0.78 |
| p_Acidobacteria | 0.0003 | 0.0011 | 0.0029 | 0.0084 | 0.0000 | 8.85 | 0.83 | 0.62 | 0.89 | 0.82 | 0.63 | 0.80 |
| p_Gemmatimonadetes | 0.0001 | 0.0002 | 0.0005 | 0.0019 | 0.0000 | 10.04 | 0.80 | 0.62 | 0.86 | 0.77 | 0.66 | 0.77 |
| p_Chloroflexi | 0.0001 | 0.0003 | 0.0022 | 0.0047 | 0.0000 | 22.41 | 0.84 | 0.62 | 0.91 | 0.87 | 0.63 | 0.89 |

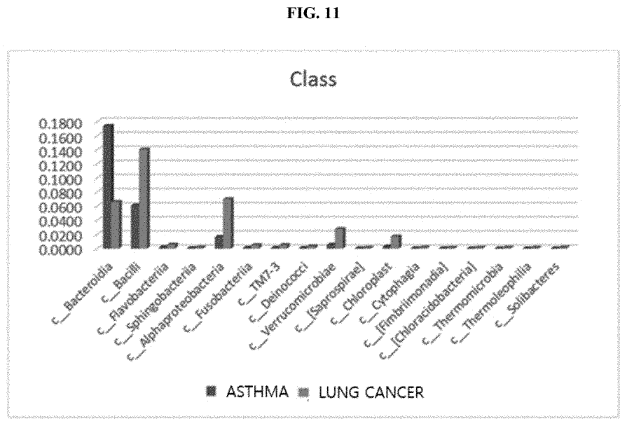

As a result of analyzing bacteria-derived extracellular vesicles in blood at a class level, a diagnostic model developed using, as a biomarker, one or more bacteria selected from the class Bacteroidia, the class Bacilli, the class Flavobacteriia, the class Sphingobacteriia, the class Alphaproteobacteria, the class Fusobacteriia, the class TM7-3, the class Deinococci, the class Verrucomicrobiae, the class Saprospirae, the class Chloroplast, the class Cytophagia, the class Fimbriimonadia, the class Chloracidobacteria, the class Thermomicrobia, the class Thermoleophilia, and the class Solibacteres exhibited significant diagnostic performance for lung cancer (see Table 11 and FIG. 11).

TABLE 11

| | Asthma | | Lung Cancer | | t-test | | Training Set | | | Test Set | | |
|---|---|---|---|---|---|---|---|---|---|---|---|---|
| Taxon | Mean | SD | Mean | SD | p-value | fold | AUC | sensitivity | specificity | AUC | sensitivity | specificity |
| c_Bacteroidia | 0.1736 | 0.0514 | 0.0661 | 0.0380 | 0.0000 | 0.38 | 0.97 | 0.89 | 0.94 | 0.95 | 0.88 | 0.94 |
| c_Bacilli | 0.0611 | 0.0408 | 0.1404 | 0.0728 | 0.0000 | 2.30 | 0.92 | 0.81 | 0.91 | 0.91 | 0.85 | 0.91 |
| c_Flavobacteriia | 0.0018 | 0.0066 | 0.0055 | 0.0088 | 0.0000 | 3.07 | 0.86 | 0.62 | 0.86 | 0.80 | 0.72 | 0.86 |
| c_Sphingobacteriia | 0.0004 | 0.0011 | 0.0014 | 0.0042 | 0.0000 | 3.74 | 0.81 | 0.62 | 0.85 | 0.76 | 0.66 | 0.85 |
| c_Alphaproteobacteria | 0.0163 | 0.0323 | 0.0700 | 0.0453 | 0.0000 | 4.29 | 0.95 | 0.82 | 0.91 | 0.92 | 0.85 | 0.91 |
| c_Fusobacteriia | 0.0009 | 0.0029 | 0.0047 | 0.0095 | 0.0000 | 5.04 | 0.85 | 0.63 | 0.89 | 0.84 | 0.68 | 0.89 |
| c_TM7-3 | 0.0009 | 0.0027 | 0.0048 | 0.0079 | 0.0000 | 5.19 | 0.87 | 0.66 | 0.90 | 0.77 | 0.68 | 0.90 |
| c_Deinococci | 0.0006 | 0.0019 | 0.0031 | 0.0065 | 0.0000 | 5.25 | 0.83 | 0.62 | 0.88 | 0.80 | 0.65 | 0.88 |
| c_Verrucomicrobiae | 0.0052 | 0.0060 | 0.0274 | 0.0235 | 0.0000 | 5.29 | 0.91 | 0.79 | 0.83 | 0.87 | 0.76 | 0.83 |
| c_[Saprospirae] | 0.0002 | 0.0006 | 0.0010 | 0.0034 | 0.0000 | 5.34 | 0.81 | 0.63 | 0.88 | 0.77 | 0.63 | 0.88 |
| c_Chloroplast | 0.0027 | 0.0049 | 0.0169 | 0.0315 | 0.0000 | 6.22 | 0.89 | 0.68 | 0.87 | 0.86 | 0.75 | 0.87 |
| c_Cylophagia | 0.0002 | 0.0008 | 0.0014 | 0.0040 | 0.0000 | 6.73 | 0.82 | 0.62 | 0.86 | 0.77 | 0.68 | 0.86 |
| c_[Fimbriimonadia] | 0.0001 | 0.0004 | 0.0009 | 0.0051 | 0.0046 | 8.81 | 0.81 | 0.62 | 0.87 | 0.76 | 0.65 | 0.87 |
| c_[Chloracidobacteria] | 0.0001 | 0.0005 | 0.0010 | 0.0052 | 0.0015 | 11.88 | 0.81 | 0.62 | 0.90 | 0.76 | 0.65 | 0.90 |
| c_Thermomicrobia | 0.0000 | 0.0002 | 0.0012 | 0.0033 | 0.0000 | 26.04 | 0.82 | 0.62 | 0.89 | 0.84 | 0.63 | 0.89 |
| c_Thermoleophilia | 0.0000 | 0.0002 | 0.0010 | 0.0064 | 0.0069 | 28.53 | 0.80 | 0.62 | 0.87 | 0.77 | 0.66 | 0.87 |
| c_Solibacteres | 0.0000 | 0.0002 | 0.0013 | 0.0051 | 0.0000 | 41.68 | 0.81 | 0.61 | 0.87 | 0.82 | 0.66 | 0.87 |

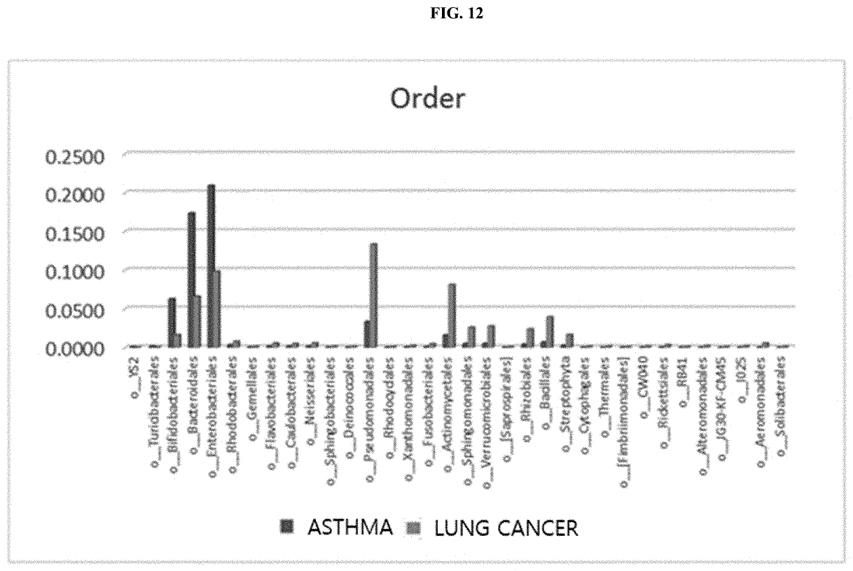

As a result of analyzing bacteria-derived extracellular vesicles in blood at an order level, a diagnostic model developed using, as a biomarker, one or more bacteria selected from the order YS2, the order Turicibacterales, the order Bifidobacteriales, the order Bacteroidales, the order Enterobacteriales, the order Rhodobacterales, the order Gemellales, the order Flavobacteriales, the order Caulobacterales, the order Neisseriales, the order Sphingobacteriales, the order Deinococcales, the order Pseudomonadales, the order Rhodocyclales, the order Xanthomonadales, the order Fusobacteriales, the order Actinomycetales, the order Sphingomonadales, the order Verrucomicrobiales, the order Saprospirales, the order Rhizobiales, the order Bacillales, the order Streptophyta, the order Cytophagales, the order Thermales, the order Fimbriimonadales, the order CW040, the order Rickettsiales, the order RB41, the order Alteromonadales, the order JG30-KF-CM45, the order 1025, the order Aeromonadales, and the order Solibacterales exhibited significant diagnostic performance for lung cancer (see Table 12 and FIG. 12).

TABLE 12

| | Asthma | | Lung Cancer | | t-test | | Training Set | | | Test Set | | |
|---|---|---|---|---|---|---|---|---|---|---|---|---|
| Taxon | Mean | SD | Mean | SD | p-value | fold | AUC | sensitivity | specificity | AUC | sensitivity | specificity |
| o_YS2 | 0.0007 | 0.0014 | 0.0001 | 0.0009 | 0.0000 | 0.16 | 0.81 | 0.64 | 0.86 | 0.81 | 0.66 | 0.75 |
| o_Turicibacterales | 0.0017 | 0.0053 | 0.0004 | 0.0017 | 0.0009 | 0.25 | 0.80 | 0.62 | 0.86 | 0.77 | 0.65 | 0.74 |
| o_Bifidobacteriales | 0.0627 | 0.0335 | 0.0164 | 0.0144 | 0.0000 | 0.26 | 0.95 | 0.85 | 0.95 | 0.92 | 0.82 | 0.88 |
| o_Bacteroidales | 0.1736 | 0.0514 | 0.0661 | 0.0380 | 0.0000 | 0.38 | 0.97 | 0.89 | 0.94 | 0.95 | 0.88 | 0.92 |
| o_Enterobacteriales | 0.2091 | 0.0878 | 0.0983 | 0.0629 | 0.0000 | 0.47 | 0.91 | 0.78 | 0.91 | 0.91 | 0.81 | 0.85 |
| o_Rhodobacterales | 0.0034 | 0.0237 | 0.0080 | 0.0125 | 0.0092 | 2.36 | 0.80 | 0.62 | 0.86 | 0.77 | 0.68 | 0.74 |
| o_Gemellales | 0.0004 | 0.0032 | 0.0012 | 0.0032 | 0.0089 | 2.64 | 0.79 | 0.62 | 0.86 | 0.76 | 0.66 | 0.75 |
| o_Flavobacteriales | 0.0018 | 0.0066 | 0.0055 | 0.0088 | 0.0000 | 3.07 | 0.86 | 0.62 | 0.86 | 0.80 | 0.72 | 0.82 |
| o_Caulobacterales | 0.0015 | 0.0030 | 0.0050 | 0.0072 | 0.0000 | 3.31 | 0.85 | 0.61 | 0.89 | 0.80 | 0.71 | 0.77 |
| o_Neisseriales | 0.0016 | 0.0064 | 0.0058 | 0.0119 | 0.0000 | 3.61 | 0.82 | 0.62 | 0.87 | 0.80 | 0.69 | 0.76 |
| o_Sphingobacteriales | 0.0004 | 0.0011 | 0.0014 | 0.0042 | 0.0000 | 3.74 | 0.81 | 0.62 | 0.85 | 0.76 | 0.66 | 0.74 |
| o_Deinococcales | 0.0004 | 0.0013 | 0.0014 | 0.0053 | 0.0010 | 3.89 | 0.79 | 0.62 | 0.86 | 0.76 | 0.66 | 0.75 |
| o_Pseudomonadales | 0.0335 | 0.0496 | 0.1334 | 0.0791 | 0.0000 | 3.98 | 0.96 | 0.87 | 0.92 | 0.93 | 0.87 | 0.86 |
| o_Rhodocyclales | 0.0003 | 0.0009 | 0.0012 | 0.0045 | 0.0006 | 4.31 | 0.80 | 0.62 | 0.86 | 0.75 | 0.65 | 0.75 |
| o_Xanthomonadales | 0.0006 | 0.0012 | 0.0025 | 0.0059 | 0.0000 | 4.47 | 0.81 | 0.62 | 0.88 | 0.77 | 0.68 | 0.76 |
| o_Fusobacteriales | 0.0009 | 0.0029 | 0.0047 | 0.0095 | 0.0000 | 5.04 | 0.85 | 0.63 | 0.89 | 0.84 | 0.68 | 0.84 |
| o_Actinomycetales | 0.0160 | 0.0218 | 0.0813 | 0.0458 | 0.0000 | 5.07 | 0.97 | 0.90 | 0.92 | 0.96 | 0.88 | 0.88 |
| o_Sphingomonadales | 0.0049 | 0.0082 | 0.0261 | 0.0254 | 0.0000 | 5.27 | 0.91 | 0.74 | 0.88 | 0.91 | 0.79 | 0.80 |
| o_Verrucomicrobiales | 0.0052 | 0.0060 | 0.0274 | 0.0235 | 0.0000 | 5.29 | 0.91 | 0.79 | 0.83 | 0.87 | 0.76 | 0.77 |
| o_[Saprospirales] | 0.0002 | 0.0006 | 0.0010 | 0.0034 | 0.0000 | 5.34 | 0.81 | 0.63 | 0.88 | 0.77 | 0.63 | 0.73 |
| o_Rhizobiales | 0.0043 | 0.0057 | 0.0237 | 0.0202 | 0.0000 | 5.54 | 0.93 | 0.80 | 0.87 | 0.90 | 0.84 | 0.85 |
| o_Bacillales | 0.0071 | 0.0085 | 0.0394 | 0.0509 | 0.0000 | 5.57 | 0.94 | 0.84 | 0.88 | 0.92 | 0.82 | 0.85 |
| o_Streptophyta | 0.0027 | 0.0048 | 0.0165 | 0.0314 | 0.0000 | 6.23 | 0.89 | 0.68 | 0.88 | 0.86 | 0.75 | 0.82 |
| o_Cytophagales | 0.0002 | 0.0008 | 0.0014 | 0.0040 | 0.0000 | 6.73 | 0.82 | 0.62 | 0.86 | 0.77 | 0.68 | 0.78 |
| o_Thermales | 0.0002 | 0.0014 | 0.0017 | 0.0039 | 0.0000 | 7.32 | 0.83 | 0.60 | 0.89 | 0.79 | 0.66 | 0.79 |
| o_[Fimbriimonadales] | 0.0001 | 0.0004 | 0.0009 | 0.0051 | 0.0046 | 8.81 | 0.81 | 0.62 | 0.87 | 0.76 | 0.65 | 0.78 |
| o_CW040 | 0.0002 | 0.0009 | 0.0016 | 0.0040 | 0.0000 | 9.42 | 0.84 | 0.62 | 0.88 | 0.76 | 0.66 | 0.75 |
| o_Rickettsiales | 0.0003 | 0.0011 | 0.0034 | 0.0089 | 0.0000 | 12.76 | 0.86 | 0.61 | 0.90 | 0.76 | 0.62 | 0.74 |
| o_RB41 | 0.0001 | 0.0005 | 0.0010 | 0.0052 | 0.0016 | 12.88 | 0.81 | 0.62 | 0.89 | 0.76 | 0.65 | 0.76 |
| o_Alteromonadales | 0.0002 | 0.0007 | 0.0022 | 0.0081 | 0.0000 | 13.64 | 0.82 | 0.62 | 0.86 | 0.79 | 0.66 | 0.78 |
| o_JG30-KF-CM45 | 0.0000 | 0.0002 | 0.0011 | 0.0033 | 0.0000 | 24.15 | 0.82 | 0.62 | 0.89 | 0.84 | 0.63 | 0.84 |
| o_I025 | 0.0001 | 0.0005 | 0.0019 | 0.0059 | 0.0000 | 27.72 | 0.83 | 0.62 | 0.88 | 0.79 | 0.66 | 0.78 |
| o_Aeromonadales | 0.0002 | 0.0007 | 0.0056 | 0.0320 | 0.0030 | 34.45 | 0.82 | 0.62 | 0.87 | 0.78 | 0.68 | 0.74 |
| o_Solibacterales | 0.0000 | 0.0002 | 0.0013 | 0.0051 | 0.0000 | 41.68 | 0.81 | 0.61 | 0.87 | 0.82 | 0.66 | 0.82 |

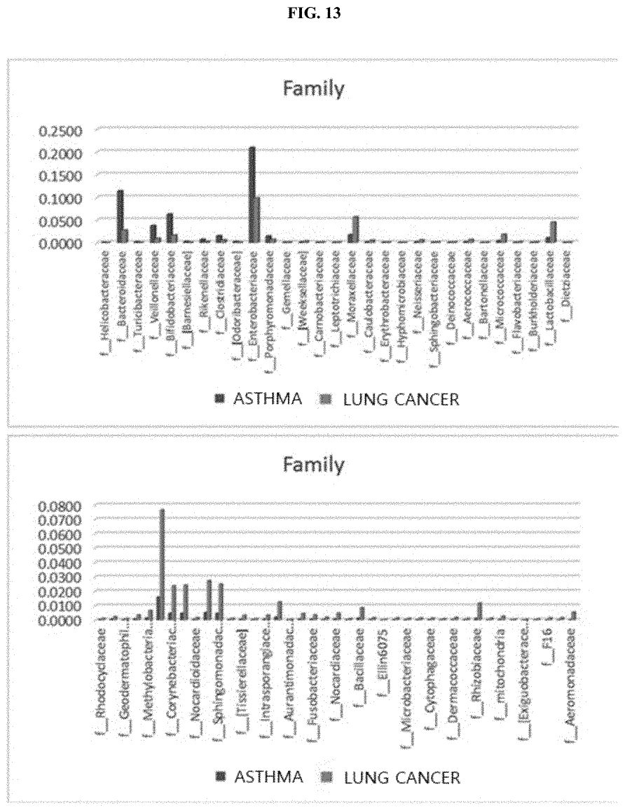

As a result of analyzing bacteria-derived extracellular vesicles in blood at a family level, a diagnostic model developed using, as a biomarker, one or more bacteria selected from the family Helicobacteraceae, the family Bacteroidaceae, the family Turicibacteraceae, the family Veillonellaceae, the family Bifidobacteriaceae, the family Barnesiellaceae, the family Rikenellaceae, the family Clostridiaceae, the family Odoribacteraceae, the family Enterobacteriaceae, the family Porphyromonadaceae, the family Gemellaceae, the family Weeksellaceae, the family Carnobacteriaceae, the family Leptotrichiaceae, the family Moraxellaceae, the family Caulobacteraceae, the family Erythrobacteraceae, the family Hyphomicrobiaceae, the family Neisseriaceae, the family Sphingobacteriaceae, the family Deinococcaceae, the family Aerococcaceae, the family Bartonellaceae, the family Micrococcaceae, the family Flavobacteriaceae, the family Burkholderiaceae, the family Lactobacillaceae, the family Dietziaceae, the family Rhodocyclaceae, the family Xanthomonadaceae, the family Geodermatophilaceae, the family Actinomycetaceae, the family Methylobacteriaceae, the family Pseudomonadaceae, the family Corynebacteriaceae, the family Staphylococcaceae, the family Nocardioidaceae, the family Verrucomicrobiaceae, the family Sphingomonadaceae, the family Mycobacteriaceae, the family Tissierellaceae, the family Chitinophagaceae, the family Intrasporangiaceae, the family Propionibacteriaceae, the family Aurantimonadaceae, the family Planococcaceae, the family Fusobacteriaceae, the family Bradyrhizobiaceae, the family Nocardiaceae, the family Dermabacteraceae, the family Bacillaceae, the family Thermaceae, the family Ellin6075, the family Brevibacteriaceae, the family Microbacteriaceae, the family Rhodospirillaceae, the family Cytophagaceae, the family Fimbriimonadaceae, the family Dermacoccaceae, the family Chromatiaceae, the family Rhizobiaceae, the family Gordoniaceae, the family mitochondria, the family Pseudonocardiaceae, the family Exiguobacteraceae, the family Shewanellaceae, the family F16, the family Rs-045, and the family Aeromonadaceae exhibited significant diagnostic performance for lung cancer (see Table 13 and FIG. 13).

TABLE 13

| | Asthma | | Lung Cancer | | t-test | | Training Set | | | Test Set | | |
|---|---|---|---|---|---|---|---|---|---|---|---|---|
| Taxon | Mean | SD | Mean | SD | p-value | fold | AUC | sensitivity | spedficity | AUC | sensitivity | specificity |
| f_Helicobacteraceae | 0.0005 | 0.0022 | 0.0000 | 0.0002 | 0.0024 | 0.05 | 0.79 | 0.64 | 0.86 | 0.77 | 0.68 | 0.75 |
| f_Bacteroidaceae | 0.1140 | 0.0469 | 0.0276 | 0.0212 | 0.0000 | 0.24 | 0.97 | 0.89 | 0.95 | 0.96 | 0.91 | 0.91 |
| f_Turicibacteraceae | 0.0017 | 0.0053 | 0.0004 | 0.0017 | 0.0009 | 0.25 | 0.80 | 0.62 | 0.86 | 0.77 | 0.65 | 0.74 |
| f_Veillonellaceae | 0.0375 | 0.0288 | 0.0094 | 0.0093 | 0.0000 | 0.25 | 0.94 | 0.79 | 0.91 | 0.93 | 0.82 | 0.88 |
| f_Bifidobacteriaceae | 0.0627 | 0.0335 | 0.0164 | 0.0144 | 0.0000 | 0.26 | 0.95 | 0.85 | 0.95 | 0.92 | 0.82 | 0.88 |
| f_[Barnesiellaceae] | 0.0024 | 0.0050 | 0.0008 | 0.0025 | 0.0000 | 0.32 | 0.80 | 0.64 | 0.85 | 0.79 | 0.65 | 0.77 |
| f_Rikenellaceae | 0.0070 | 0.0087 | 0.0025 | 0.0050 | 0.0000 | 0.36 | 0.83 | 0.68 | 0.84 | 0.81 | 0.68 | 0.79 |
| f_Clostridiaceae | 0.0151 | 0.0143 | 0.0059 | 0.0078 | 0.0000 | 0.39 | 0.82 | 0.66 | 0.86 | 0.81 | 0.69 | 0.82 |
| f_[Odoribacteraceae] | 0.0021 | 0.0029 | 0.0009 | 0.0022 | 0.0000 | 0.43 | 0.80 | 0.67 | 0.86 | 0.78 | 0.65 | 0.75 |
| f_Enterobacteriaceae | 0.2091 | 0.0878 | 0.0983 | 0.0629 | 0.0000 | 0.47 | 0.91 | 0.78 | 0.91 | 0.91 | 0.81 | 0.85 |
| f_Porphyromonadaceae | 0.0145 | 0.0090 | 0.0069 | 0.0105 | 0.0000 | 0.47 | 0.85 | 0.68 | 0.85 | 0.83 | 0.72 | 0.80 |
| f_Gemellaceae | 0.0004 | 0.0032 | 0.0012 | 0.0032 | 0.0093 | 2.65 | 0.79 | 0.62 | 0.86 | 0.76 | 0.66 | 0.75 |
| f_[Weeksellaeae] | 0.0013 | 0.0061 | 0.0036 | 0.0072 | 0.0001 | 2.68 | 0.82 | 0.61 | 0.89 | 0.78 | 0.66 | 0.80 |
| f_Carnobacteriaceae | 0.0003 | 0.0012 | 0.0010 | 0.0029 | 0.0002 | 3.14 | 0.79 | 0.62 | 0.86 | 0.76 | 0.66 | 0.77 |
| f_Leptotrichiaceae | 0.0004 | 0.0011 | 0.0012 | 0.0029 | 0.0000 | 3.23 | 0.81 | 0.62 | 0.89 | 0.77 | 0.62 | 0.79 |
| f_Moraxellaceae | 0.0175 | 0.0451 | 0.0567 | 0.0481 | 0.0000 | 3.25 | 0.90 | 0.68 | 0.89 | 0.86 | 0.74 | 0.82 |
| f_Caulobacteraceae | 0.0015 | 0.0030 | 0.0050 | 0.0072 | 0.0000 | 3.31 | 0.85 | 0.61 | 0.89 | 0.80 | 0.71 | 0.77 |
| f_Erythrobacteraceae | 0.0003 | 0.0028 | 0.0010 | 0.0045 | 0.0235 | 3.36 | 0.79 | 0.62 | 0.86 | 0.75 | 0.66 | 0.74 |
| f_Hyphomicrobiaceae | 0.0001 | 0.0006 | 0.0004 | 0.005 | 0.0020 | 3.36 | 0.79 | 0.62 | 0.87 | 0.76 | 0.65 | 0.74 |
| f_Neisseriaceae | 0.0016 | 0.0064 | 0.0058 | 0.0119 | 0.0000 | 3.61 | 0.82 | 0.62 | 0.87 | 0.80 | 0.69 | 0.76 |
| f_Sphingobacteriaceae | 0.0003 | 0.0010 | 0.0013 | 0.0041 | 0.0000 | 3.91 | 0.80 | 0.61 | 0.86 | 0.76 | 0.65 | 0.74 |
| f_Deinococcaceae | 0.0003 | 0.0013 | 0.0014 | 0.0053 | 0.0012 | 4.00 | 0.79 | 0.62 | 0.86 | 0.76 | 0.66 | 0.75 |
| f_Aerococcaceae | 0.0017 | 0.0042 | 0.0067 | 0.0107 | 0.0000 | 4.02 | 0.84 | 0.63 | 0.87 | 0.83 | 0.72 | 0.79 |
| f_Bartonellaeeae | 0.0002 | 0.0007 | 0.0009 | 0.0033 | 0.0004 | 4.06 | 0.80 | 0.62 | 0.87 | 0.76 | 0.65 | 0.74 |
| f_Micrococcaceae | 0.0045 | 0.0143 | 0.0183 | 0.0172 | 0.0000 | 4.07 | 0.91 | 0.65 | 0.89 | 0.87 | 0.76 | 0.79 |
| f_Flavobacteriaceae | 0.0005 | 0.0022 | 0.0019 | 0.0049 | 0.0000 | 4.15 | 0.83 | 0.61 | 0.87 | 0.78 | 0.68 | 0.77 |
| f_Burkholderiaceae | 0.0004 | 0.0010 | 0.0017 | 0.0064 | 0.0005 | 4.16 | 0.79 | 0.62 | 0.86 | 0.77 | 0.66 | 0.76 |
| f_Lactobacillaceae | 0.0108 | 0.0141 | 0.0454 | 0.0454 | 0.0000 | 4.21 | 0.91 | 0.80 | 0.86 | 0.89 | 0.85 | 0.82 |
| f_Dietziaceae | 0.0003 | 0.0008 | 0.0011 | 0.0049 | 0.0031 | 4.25 | 0.79 | 0.62 | 0.87 | 0.76 | 0.66 | 0.76 |
| f_Rhodocyclaceae | 0.0003 | 0.0009 | 0.0012 | 0.0045 | 0.0006 | 4.31 | 0.80 | 0.62 | 0.86 | 0.75 | 0.65 | 0.75 |
| f_Xanthomonadaceae | 0.0005 | 0.0012 | 0.0023 | 0.0058 | 0.0000 | 4.36 | 0.81 | 0.62 | 0.88 | 0.77 | 0.66 | 0.76 |
| f_Geodermatophilaceae | 0.0003 | 0.0012 | 0.0012 | 0.0034 | 0.0000 | 4.44 | 0.80 | 0.61 | 0.86 | 0.79 | 0.65 | 0.77 |
| f_Actinomycetaceae | 0.0008 | 0.0038 | 0.0035 | 0.0060 | 0.0000 | 4.46 | 0.83 | 0.62 | 0.89 | 0.82 | 0.65 | 0.79 |
| f_Methylobacteriaceae | 0.0015 | 0.0034 | 0.0066 | 0.0083 | 0.0000 | 4.49 | 0.86 | 0.64 | 0.86 | 0.81 | 0.69 | 0.74 |
| f_Pseudomonadaceae | 0.0160 | 0.0154 | 0.0765 | 0.0531 | 0.0000 | 4.77 | 0.97 | 0.91 | 0.95 | 0.95 | 0.93 | 0.88 |
| f_Corynebacteriaceae | 0.0048 | 0.0055 | 0.0238 | 0.0241 | 0.0000 | 4.95 | 0.91 | 0.79 | 0.85 | 0.90 | 0.84 | 0.80 |
| f_Staphylococcaceae | 0.0047 | 0.0068 | 0.0242 | 0.0437 | 0.0000 | 5.15 | 0.90 | 0.73 | 0.87 | 0.90 | 0.75 | 0.84 |
| f_Nocardioidaceae | 0.0003 | 0.0008 | 0.0017 | 0.0046 | 0.0000 | 5.16 | 0.81 | 0.61 | 0.85 | 0.77 | 0.68 | 0.74 |
| f_Verrucomicrobiaceae | 0.0052 | 0.0060 | 0.0274 | 0.0235 | 0.0000 | 5.29 | 0.91 | 0.79 | 0.83 | 0.87 | 0.76 | 0.77 |
| f_Sphingomonadaceae | 0.0046 | 0.0070 | 0.0249 | 0.0245 | 0.0000 | 5.39 | 0.91 | 0.74 | 0.87 | 0.91 | 0.81 | 0.82 |
| f_Mycobacteriaceae | 0.0002 | 0.0009 | 0.0012 | 0.0054 | 0.0023 | 5.60 | 0.79 | 0.62 | 0.87 | 0.77 | 0.63 | 0.76 |
| f_[Tissierellaceae] | 0.0006 | 0.0016 | 0.0033 | 0.0069 | 0.0000 | 5.62 | 0.83 | 0.60 | 0.88 | 0.80 | 0.65 | 0.82 |
| f_Chitinophagaceae | 0.0002 | 0.0006 | 0.0010 | 0.0034 | 0.0000 | 5.63 | 0.81 | 0.63 | 0.88 | 0.76 | 0.63 | 0.73 |
| f_Intrasporangiaceae | 0.0006 | 0.0013 | 0.0034 | 0.0065 | 0.0000 | 5.74 | 0.84 | 0.62 | 0.86 | 0.80 | 0.71 | 0.76 |
| f_Propionibacteriaceae | 0.0021 | 0.0031 | 0.0126 | 0.0117 | 0.0000 | 5.85 | 0.91 | 0.75 | 0.87 | 0.93 | 0.79 | 0.86 |
| f_Aurantimonadaceae | 0.0001 | 0.0003 | 0.0005 | 0.0020 | 0.0016 | 6.06 | 0.79 | 0.62 | 0.86 | 0.76 | 0.66 | 0.74 |
| f_Planococcaceae | 0.0007 | 0.0013 | 0.0045 | 0.0108 | 0.0000 | 6.20 | 0.87 | 0.64 | 0.85 | 0.83 | 0.66 | 0.83 |
| f_Fusobacteraceae | 0.0006 | 0.0026 | 0.0035 | 0.0090 | 0.0000 | 6.21 | 0.83 | 0.62 | 0.88 | 0.83 | 0.68 | 0.85 |
| f_Bradyrhizobiaceae | 0.0003 | 0.0008 | 0.0020 | 0.0054 | 0.0000 | 6.25 | 0.82 | 0.60 | 0.86 | 0.77 | 0.65 | 0.77 |
| f_Nocardiaceae | 0.0008 | 0.0019 | 0.0048 | 0.0103 | 0.0000 | 6.32 | 0.86 | 0.65 | 0.87 | 0.82 | 0.74 | 0.82 |
| f_Dermabacteraceae | 0.0002 | 0.0005 | 0.0010 | 0.0030 | 0.0000 | 6.38 | 0.80 | 0.61 | 0.88 | 0.77 | 0.65 | 0.76 |
| f_Bacillaceae | 0.0013 | 0.0020 | 0.0085 | 0.0149 | 0.0000 | 6.44 | 0.88 | 0.64 | 0.88 | 0.82 | 0.72 | 0.75 |
| f_Thermaceae | 0.0002 | 0.0014 | 0.0017 | 0.0039 | 0.0000 | 7.32 | 0.83 | 0.60 | 0.89 | 0.79 | 0.66 | 0.79 |
| f_Ellin6075 | 0.0001 | 0.0004 | 0.0005 | 0.0018 | 0.0001 | 7.54 | 0.80 | 0.62 | 0.89 | 0.76 | 0.65 | 0.77 |
| f_Brevibacteriaceae | 0.0002 | 0.0006 | 0.0015 | 0.0061 | 0.0002 | 7.65 | 0.81 | 0.62 | 0.89 | 0.77 | 0.66 | 0.76 |
| f_Microbacteriaceae | 0.0002 | 0.0008 | 0.0013 | 0.0038 | 0.0000 | 7.69 | 0.82 | 0.62 | 0.86 | 0.79 | 0.68 | 0.77 |
| f_Rhodospirillaceae | 0.0002 | 0.0011 | 0.0015 | 0.0062 | 0.0002 | 7.94 | 0.79 | 0.62 | 0.87 | 0.77 | 0.66 | 0.76 |
| f_Cytophagaceae | 0.0002 | 0.0005 | 0.0013 | 0.0040 | 0.0000 | 8.57 | 0.82 | 0.61 | 0.87 | 0.77 | 0.63 | 0.78 |
| f_[Fimbriimonadaceae] | 0.0001 | 0.0004 | 0.0009 | 0.0051 | 0.0048 | 8.78 | 0.81 | 0.62 | 0.87 | 0.76 | 0.65 | 0.78 |
| f_Dermacoccaceae | 0.0002 | 0.0005 | 0.0019 | 0.0048 | 0.0000 | 9.05 | 0.83 | 0.63 | 0.88 | 0.80 | 0.69 | 0.78 |
| f_[Chromatiaceae] | 0.0001 | 0.0006 | 0.0011 | 0.0062 | 0.0056 | 9.57 | 0.80 | 0.62 | 0.87 | 0.75 | 0.66 | 0.74 |
| f_Rhizobiaceae | 0.0012 | 0.0025 | 0.0118 | 0.0150 | 0.0000 | 9.97 | 0.92 | 0.76 | 0.88 | 0.90 | 0.76 | 0.83 |
| f_Gordoniaceae | 0.0001 | 0.0005 | 0.0013 | 0.0048 | 0.0000 | 10.20 | 0.80 | 0.62 | 0.85 | 0.77 | 0.65 | 0.76 |
| f_mitochondria | 0.0003 | 0.0011 | 0.0027 | 0.0081 | 0.0000 | 10.41 | 0.84 | 0.60 | 0.89 | 0.75 | 0.60 | 0.74 |
| f_Pseudonocardiaceae | 0.0001 | 0.0002 | 0.0006 | 0.0025 | 0.0001 | 11.72 | 0.80 | 0.63 | 0.86 | 0.76 | 0.63 | 0.75 |
| f_[Exiguobacteraceae] | 0.0000 | 0.0002 | 0.0006 | 0.0033 | 0.0048 | 23.39 | 0.80 | 0.62 | 0.86 | 0.77 | 0.65 | 0.79 |
| f_Shewanellaceae | 0.0000 | 0.0004 | 0.0009 | 0.0040 | 0.0003 | 24.54 | 0.80 | 0.62 | 0.86 | 0.77 | 0.66 | 0.77 |
| f_F16 | 0.0001 | 0.0005 | 0.0016 | 0.0040 | 0.0000 | 24.68 | 0.85 | 0.63 | 0.88 | 0.78 | 0.68 | 0.74 |
| f_Rs-045 | 0.0001 | 0.0005 | 0.0017 | 0.0058 | 0.0000 | 26.38 | 0.83 | 0.62 | 0.90 | 0.78 | 0.65 | 0.78 |
| f_Aeromonadaceae | 0.0002 | 0.0007 | 0.0056 | 0.0320 | 0.0030 | 34.42 | 0.82 | 0.62 | 0.87 | 0.78 | 0.68 | 0.74 |

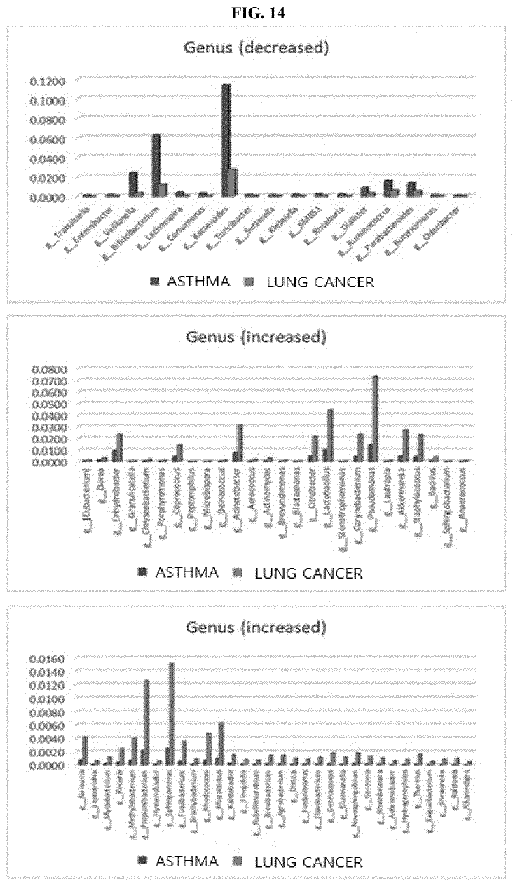

As a result of analyzing bacteria-derived extracellular vesicles in blood at a genus level, a diagnostic model developed using, as a biomarker, one or more bacteria selected from of the genus Trabulsiella, the genus Enterobacter, the genus Veillonella, the genus Bifidobacterium, the genus Lachnospira, the genus Comamonas, the genus Bacteroides, the genus Turicibacter, the genus Sutterella, the genus Klebsiella, the genus SMB53, the genus Roseburia, the genus Dialister, the genus Ruminococcus, the genus Parabacteroides, the genus Butyricimonas, the genus Odoribacter, the genus Eubacterium, the genus Dorea, the genus Enhydrobacter, the genus Granulicatella, the genus Chryseobacterium, the genus Porphyromonas, the genus Coprococcus, the genus Peptomphilus, the genus Microbispora, the genus Deinococcus, the genus Acinetobacter, the genus Aerococcus, the genus Actinomyces, the genus Brevundimonas, the genus Blastomonas, the genus Citrobacter, the genus Lactobacillus, the genus Stenotrophomonas, the genus Corynebacterium, the genus Pseudomonas, the genus Lautropia, the genus Akkermansia, the genus Staphylococcus, the genus Bacillus, the genus Sphingobacterium, the genus Anaerococcus, the genus Neisseria, the genus Leptotrichia, the genus Mycobacterium, the genus Kocuria, the genus Methylobacterium, the genus Propionibacterium, the genus Hymenobacter, the genus Sphingomonas, the genus Fusobacterium, the genus Brachybacterium, the genus Rhodococcus, the genus Micrococcus, the genus Kaistobacter, the genus Finegoldia, the genus Rubellimicrobium, the genus Brevibacterium, the genus Agrobacterium, the genus Dietzia, the genus Fimbriimonas, the genus Flavobacterium, the genus Dermacoccus, the genus Skermanella, the genus Novosphingobium, the genus Gordonia, the genus Rheinheimera, the genus Achromobacter, the genus Hydrogenophilus, the genus Thermus, the genus Exiguobacterium, the genus Shewanella, the genus Ralstonia, and the genus Alkanindiges exhibited significant diagnostic performance for lung cancer (see Table 14 and FIG. 14).

TABLE 14

| | Asthma | | Lung Cancer | | t-test | | Training Set | | | Test Set | | |
|---|---|---|---|---|---|---|---|---|---|---|---|---|
| Taxon | Mean | SD | Mean | SD | p-value | fold | AUC | sensitivity | specificity | AUC | sensitivity | specificity |
| g_Trabulsiella | 0.0007 | 0.0012 | 0.0000 | 0.0002 | 0.0000 | 0.04 | 0.92 | 0.75 | 0.92 | 0.91 | 0.82 | 0.89 |
| g_Enterobacter | 0.0016 | 0.0013 | 0.0002 | 0.0018 | 0.0000 | 0.11 | 0.93 | 0.77 | 0.93 | 0.91 | 0.79 | 0.87 |
| g_Veillonella | 0.0243 | 0.0248 | 0.0035 | 0.0054 | 0.0000 | 0.14 | 0.94 | 0.82 | 0.91 | 0.93 | 0.84 | 0.87 |
| g_Bifidobacterium | 0.0623 | 0.0336 | 0.0123 | 0.0120 | 0.0000 | 0.20 | 0.96 | 0.87 | 0.96 | 0.95 | 0.82 | 0.92 |
| g_Lachnospira | 0.0037 | 0.0130 | 0.0008 | 0.0022 | 0.0013 | 0.23 | 0.86 | 0.67 | 0.87 | 0.82 | 0.72 | 0.78 |
| g_Comamonas | 0.0031 | 0.0091 | 0.0007 | 0.0023 | 0.0002 | 0.23 | 0.81 | 0.63 | 0.86 | 0.80 | 0.69 | 0.78 |
| g_Bacteroides | 0.1140 | 0.0469 | 0.0276 | 0.0212 | 0.0000 | 0.24 | 0.97 | 0.89 | 0.95 | 0.96 | 0.91 | 0.91 |
| g_Turicibacter | 0.0017 | 0.0053 | 0.0004 | 0.0017 | 0.0009 | 0.25 | 0.80 | 0.62 | 0.86 | 0.77 | 0.65 | 0.74 |
| g_Sutterella | 0.0014 | 0.0029 | 0.0004 | 0.0013 | 0.0000 | 0.27 | 0.80 | 0.66 | 0.84 | 0.77 | 0.62 | 0.77 |
| g_Klebsiella | 0.0018 | 0.0012 | 0.0006 | 0.0020 | 0.0000 | 0.31 | 0.90 | 0.72 | 0.87 | 0.88 | 0.79 | 0.79 |
| g_SMB53 | 0.0023 | 0.0033 | 0.0007 | 0.0015 | 0.0000 | 0.32 | 0.81 | 0.63 | 0.86 | 0.78 | 0.69 | 0.74 |
| g_Roseburia | 0.0021 | 0.0029 | 0.0007 | 0.0031 | 0.0000 | 0.33 | 0.80 | 0.64 | 0.86 | 0.78 | 0.65 | 0.76 |
| g_Dialister | 0.0090 | 0.0077 | 0.0033 | 0.0047 | 0.0000 | 0.37 | 0.86 | 0.67 | 0.87 | 0.86 | 0.76 | 0.78 |
| g_Ruminococcus | 0.0163 | 0.0112 | 0.0062 | 0.0102 | 0.0000 | 0.38 | 0.89 | 0.72 | 0.88 | 0.84 | 0.76 | 0.78 |
| g_Parabacteroides | 0.0138 | 0.0086 | 0.0055 | 0.0099 | 0.0000 | 0.40 | 0.87 | 0.70 | 0.87 | 0.86 | 0.75 | 0.86 |
| g_Butyricimonas | 0.0012 | 0.0019 | 0.0005 | 0.0016 | 0.0000 | 0.42 | 0.80 | 0.64 | 0.85 | 0.78 | 0.63 | 0.74 |
| g_Odoribacter | 0.0009 | 0.0017 | 0.0004 | 0.0015 | 0.0008 | 0.46 | 0.80 | 0.64 | 0.87 | 0.76 | 0.65 | 0.76 |
| g_[Eubacterium] | 0.0007 | 0.0013 | 0.0015 | 0.0028 | 0.0000 | 2.09 | 0.81 | 0.61 | 0.87 | 0.76 | 0.63 | 0.76 |
| g_Dorea | 0.0017 | 0.0072 | 0.0037 | 0.0061 | 0.0009 | 2.18 | 0.82 | 0.61 | 0.87 | 0.79 | 0.69 | 0.78 |
| g_Enhydrobacter | 0.0093 | 0.0425 | 0.0236 | 0.0293 | 0.0000 | 2.54 | 0.84 | 0.62 | 0.86 | 0.78 | 0.68 | 0.75 |
| g_Granulicatelia | 0.0003 | 0.0011 | 0.0007 | 0.0024 | 0.0051 | 2.58 | 0.79 | 0.62 | 0.86 | 0.75 | 0.66 | 0.76 |
| g_Chryseobacterium | 0.0007 | 0.0025 | 0.0019 | 0.0042 | 0.0000 | 2.74 | 0.80 | 0.62 | 0.88 | 0.77 | 0.66 | 0.76 |
| g_Porphyromonas | 0.0005 | 0.0036 | 0.0013 | 0.0033 | 0.0065 | 2.78 | 0.79 | 0.62 | 0.86 | 0.76 | 0.66 | 0.75 |
| g_Coprococcus | 0.0047 | 0.0050 | 0.0141 | 0.0146 | 0.0000 | 2.97 | 0.85 | 0.64 | 0.89 | 0.82 | 0.71 | 0.79 |
| g_Peptoniphilus | 0.0001 | 0.0005 | 0.0004 | 0.0013 | 0.0012 | 3.17 | 0.79 | 0.61 | 0.86 | 0.76 | 0.66 | 0.76 |
| g_Microbispora | 0.0002 | 0.0006 | 0.0006 | 0.0024 | 0.0042 | 3.57 | 0.79 | 0.62 | 0.86 | 0.75 | 0.65 | 0.75 |
| g_Deinococcus | 0.0003 | 0.0013 | 0.0013 | 0.0052 | 0.0020 | 3.81 | 0.79 | 0.62 | 0.86 | 0.76 | 0.66 | 0.75 |
| g_Acinetobacter | 0.0076 | 0.0128 | 0.0310 | 0.0278 | 0.0000 | 4.09 | 0.91 | 0.78 | 0.84 | 0.89 | 0.78 | 0.85 |
| g_Aerococcus | 0.0005 | 0.0020 | 0.0022 | 0.0075 | 0.0002 | 4.10 | 0.79 | 0.62 | 0.86 | 0.77 | 0.66 | 0.77 |
| g_Actinomyces | 0.0008 | 0.0038 | 0.0032 | 0.0057 | 0.0000 | 4.12 | 0.82 | 0.62 | 0.89 | 0.81 | 0.65 | 0.78 |
| g_Brevundimonas | 0.0003 | 0.0008 | 0.0012 | 0.0034 | 0.0000 | 4.24 | 0.80 | 0.61 | 0.87 | 0.78 | 0.66 | 0.76 |
| g_Blastomonas | 0.0002 | 0.0016 | 0.0007 | 0.0024 | 0.0022 | 4.33 | 0.80 | 0.62 | 0.86 | 0.77 | 0.66 | 0.76 |
| g_Citrobacter | 0.0050 | 0.0041 | 0.0218 | 0.0220 | 0.0000 | 4.34 | 0.87 | 0.65 | 0.87 | 0.85 | 0.75 | 0.79 |
| g_Lactobacillus | 0.0101 | 0.0140 | 0.0444 | 0.0454 | 0.0000 | 4.39 | 0.91 | 0.79 | 0.85 | 0.89 | 0.85 | 0.79 |
| g_Stenotrophomonas | 0.0001 | 0.0005 | 0.0005 | 0.0019 | 0.0005 | 4.58 | 0.79 | 0.62 | 0.86 | 0.75 | 0.65 | 0.77 |
| g_Corynebacterium | 0.0048 | 0.0055 | 0.0238 | 0.0241 | 0.0000 | 4.95 | 0.91 | 0.79 | 0.85 | 0.90 | 0.84 | 0.80 |
| g_Pseudomonas | 0.0144 | 0.0145 | 0.0734 | 0.0506 | 0.0000 | 5.11 | 0.97 | 0.91 | 0.95 | 0.96 | 0.93 | 0.88 |
| g_Lautropia | 0.0002 | 0.0005 | 0.0013 | 0.0060 | 0.0027 | 5.15 | 0.79 | 0.62 | 0.86 | 0.77 | 0.66 | 0.75 |
| g_Akkermansia | 0.0052 | 0.0060 | 0.0273 | 0.0236 | 0.0000 | 5.27 | 0.91 | 0.78 | 0.82 | 0.87 | 0.78 | 0.77 |
| g_Staphylococcus | 0.0044 | 0.0062 | 0.0233 | 0.0435 | 0.0000 | 5.29 | 0.90 | 0.72 | 0.89 | 0.90 | 0.76 | 0.83 |
| g_Bacillus | 0.0008 | 0.0016 | 0.0042 | 0.0108 | 0.0000 | 5.34 | 0.84 | 0.62 | 0.89 | 0.78 | 0.66 | 0.80 |
| g_Sphingobacterium | 0.0001 | 0.0006 | 0.0005 | 0.0019 | 0.0011 | 5.39 | 0.79 | 0.61 | 0.86 | 0.76 | 0.66 | 0.75 |
| g_Anaerococcus | 0.0003 | 0.0013 | 0.0014 | 0.0041 | 0.0000 | 5.39 | 0.80 | 0.62 | 0.86 | 0.77 | 0.63 | 0.78 |
| g_Neisseria | 0.0008 | 0.0026 | 0.0041 | 0.0102 | 0.0000 | 5.46 | 0.83 | 0.62 | 0.87 | 0.80 | 0.68 | 0.78 |
| g_Leptotrichia | 0.0001 | 0.0005 | 0.0006 | 0.0021 | 0.0000 | 5.54 | 0.80 | 0.62 | 0.87 | 0.76 | 0.65 | 0.78 |
| g_Mycobacterium | 0.0002 | 0.0009 | 0.0012 | 0.0054 | 0.0023 | 5.60 | 0.79 | 0.62 | 0.87 | 0.77 | 0.63 | 0.76 |
| g_Kocuria | 0.0004 | 0.0014 | 0.0025 | 0.0058 | 0.0000 | 5.66 | 0.83 | 0.62 | 0.88 | 0.80 | 0.68 | 0.82 |
| g_Methylobacterium | 0.0007 | 0.0020 | 0.0040 | 0.0068 | 0.0000 | 5.70 | 0.84 | 0.62 | 0.86 | 0.79 | 0.69 | 0.78 |
| g_Propionibacterium | 0.0021 | 0.0031 | 0.0125 | 0.0117 | 0.0000 | 5.84 | 0.91 | 0.75 | 0.87 | 0.93 | 0.79 | 0.85 |
| g_Hymenobacter | 0.0001 | 0.0004 | 0.0006 | 0.0021 | 0.0001 | 5.97 | 0.80 | 0.60 | 0.86 | 0.75 | 0.66 | 0.75 |
| g_Sphingomonas | 0.0025 | 0.0040 | 0.0151 | 0.0166 | 0.0000 | 5.99 | 0.90 | 0.68 | 0.90 | 0.89 | 0.81 | 0.78 |
| g_Fusobacterium | 0.0006 | 0.0026 | 0.0035 | 0.0090 | 0.0000 | 6.21 | 0.83 | 0.62 | 0.88 | 0.83 | 0.68 | 0.85 |
| g_Brachybacterium | 0.0001 | 0.0005 | 0.0009 | 0.0030 | 0.0000 | 6.22 | 0.80 | 0.62 | 0.87 | 0.76 | 0.66 | 0.75 |
| g_Rhodococcus | 0.0008 | 0.0019 | 0.0047 | 0.0102 | 0.0000 | 6.25 | 0.86 | 0.65 | 0.87 | 0.81 | 0.74 | 0.80 |
| g_Micrococcus | 0.0010 | 0.0017 | 0.0063 | 0.0098 | 0.0000 | 6.46 | 0.86 | 0.64 | 0.88 | 0.81 | 0.74 | 0.74 |
| g_Kaistobacter | 0.0002 | 0.0010 | 0.0016 | 0.0055 | 0.0000 | 6.46 | 0.81 | 0.61 | 0.88 | 0.76 | 0.65 | 0.74 |
| g_Finegoldia | 0.0001 | 0.0006 | 0.0008 | 0.0035 | 0.0003 | 7.22 | 0.80 | 0.62 | 0.87 | 0.77 | 0.65 | 0.76 |
| g_Rubellimicrobium | 0.0001 | 0.0007 | 0.0007 | 0.0040 | 0.0074 | 7.31 | 0.79 | 0.62 | 0.86 | 0.76 | 0.66 | 0.74 |
| g_Brevibacterium | 0.0002 | 0.0006 | 0.0015 | 0.0061 | 0.0002 | 7.65 | 0.81 | 0.62 | 0.89 | 0.77 | 0.66 | 0.76 |
| g_Agrobacterium | 0.0002 | 0.0007 | 0.0015 | 0.0036 | 0.0000 | 7.73 | 0.82 | 0.63 | 0.88 | 0.79 | 0.65 | 0.78 |
| g_Dietzia | 0.0001 | 0.0006 | 0.0010 | 0.0048 | 0.0017 | 8.13 | 0.80 | 0.63 | 0.87 | 0.76 | 0.66 | 0.76 |
| g_Fimbriimonas | 0.0001 | 0.0004 | 0.0009 | 0.0050 | 0.0060 | 8.88 | 0.81 | 0.62 | 0.87 | 0.76 | 0.65 | 0.78 |
| g_Flavobacterium | 0.0001 | 0.0005 | 0.0012 | 0.0040 | 0.0000 | 8.98 | 0.82 | 0.61 | 0.86 | 0.77 | 0.68 | 0.76 |
| g_Dermacoccus | 0.0002 | 0.0005 | 0.0019 | 0.0048 | 0.0000 | 9.05 | 0.83 | 0.63 | 0.88 | 0.80 | 0.69 | 0.78 |
| g_Skermanella | 0.0001 | 0.0009 | 0.0012 | 0.0058 | 0.0020 | 9.54 | 0.79 | 0.62 | 0.87 | 0.77 | 0.66 | 0.76 |
| g_Novosphingobium | 0.0002 | 0.0009 | 0.0019 | 0.0043 | 0.0000 | 9.63 | 0.83 | 0.62 | 0.86 | 0.80 | 0.66 | 0.79 |
| g_Gordonia | 0.0001 | 0.0005 | 0.0013 | 0.0048 | 0.0000 | 10.20 | 0.80 | 0.62 | 0.85 | 0.77 | 0.65 | 0.76 |
| g_Rheinheimera | 0.0001 | 0.0005 | 0.0010 | 0.0062 | 0.0075 | 11.51 | 0.79 | 0.63 | 0.87 | 0.76 | 0.66 | 0.74 |
| g_Achromobacter | 0.0001 | 0.0004 | 0.0006 | 0.0028 | 0.0003 | 12.39 | 0.81 | 0.62 | 0.88 | 0.75 | 0.63 | 0.76 |
| g_Hydrogenophilus | 0.0001 | 0.0007 | 0.0009 | 0.0041 | 0.0011 | 12.54 | 0.79 | 0.62 | 0.86 | 0.76 | 0.66 | 0.75 |
| g_Thermus | 0.0001 | 0.0007 | 0.0017 | 0.0038 | 0.0000 | 16.82 | 0.83 | 0.61 | 0.88 | 0.80 | 0.66 | 0.79 |
| g_Exiguobacterium | 0.0000 | 0.0002 | 0.0006 | 0.0033 | 0.0049 | 23.75 | 0.80 | 0.62 | 0.86 | 0.77 | 0.65 | 0.79 |
| g_Shewanella | 0.0000 | 0.0004 | 0.0009 | 0.0040 | 0.0003 | 24.61 | 0.80 | 0.62 | 0.86 | 0.77 | 0.66 | 0.77 |
| g_Ralstonia | 0.0000 | 0.0001 | 0.0010 | 0.0033 | 0.0000 | 28.31 | 0.82 | 0.62 | 0.88 | 0.80 | 0.66 | 0.79 |
| g_Alkanindiges | 0.0000 | 0.0001 | 0.0005 | 0.0034 | 0.0095 | 40.34 | 0.80 | 0.62 | 0.86 | 0.74 | 0.66 | 0.74 |

The above description of the present invention is provided only for illustrative purposes, and it will be understood by one of ordinary skill in the art to which the present invention pertains that the invention may be embodied in various modified forms without departing from the spirit or essential characteristics thereof. Thus, the embodiments described herein should be considered in an illustrative sense only and not for the purpose of limitation.

### INDUSTRIAL APPLICABILITY

According to the present invention, a risk group for lung cancer can be diagnosed early and predicted by predicting a risk for lung cancer through metagenomic analysis of bacteria or bacteria-derived extracellular vesicles from a human body-derived sample, and thus the onset of lung cancer can be delayed or lung cancer may be prevented through appropriate management, and, even after lung cancer occurs, early diagnosis for lung cancer can be implemented, thereby lowering a disease rate and increasing therapeutic effects. In addition, patients diagnosed with lung cancer are able to avoid exposure to causative factors predicted by metagenomic analysis, whereby the progression of lung cancer is ameliorated, or recurrence of lung cancer can be prevented.

1. A method of providing information for lung cancer diagnosis, the method comprising the following processes:
(a) extracting DNA from extracellular vesicles isolated from a subject sample;
(b) performing polymerase chain reaction (PCR) on the extracted DNA using a pair of primers having SEQ ID NO:1 and SEQ ID NO: 2; and
(c) comparing an increase or decrease in content of bacteria-derived extracellular vesicles of the subject sample with that of a normal individual-derived sample, a chronic obstructive pulmonary disease patient-derived sample, or an asthma patient-derived sample through sequencing of a product of the PCR.

2. The method of claim 1, wherein the comparing comprises at least one selected from the group consisting of:
comparing an increase or decrease in content of extracellular vesicles derived from one or more bacteria selected from the group consisting of the phylum Acidobacteria and the phylum Chloroflexi with that of the normal individual-derived sample;
comparing an increase or decrease in content of extracellular vesicles derived from one or more bacteria selected from the group consisting of the class Thermomicrobia and the class Solibacteres with that of the normal individual-derived sample;
comparing an increase or decrease in content of extracellular vesicles derived from one or more bacteria selected from the group consisting of the order Turicibacterales, the order Rickettsiales, the order Alteromonadales, the order RF32, the order Thermales, the order JG30-KF-CM45, the order 1025, the order Solibacterales, and the order Aeromonadales with that of the normal individual-derived sample;
comparing an increase or decrease in content of extracellular vesicles derived from one or more bacteria selected from the group consisting of the family Turicibacteraceae, the family Clostridiaceae, the family S24-7, the family Rhizobiaceae, the family mitochondria, the family F16, the family Gordoniaceae, the family Rhodospirillaceae, the family Thermaceae, the family Shewanellaceae, the family Ellin6075, the family Rs-045, and the family Aeromonadaceae with that of the normal individual-derived sample;
comparing an increase or decrease in content of extracellular vesicles derived from one or more bacteria selected from the group consisting of the genus Chromohalobacter, the genus Geobacillus, the genus Proteus, the genus Megamonas, the genus Moraxella, the genus Alloiococcus, the genus Turicibacter, the genus SMB53, the genus Veillonella, the genus Peptomphilus, the genus Comamonas, the genus Hymenobacter, the genus Citrobacter, the genus Novosphingobium, the genus Gordonia, the genus Aerococcus, the genus Thermus, the genus Shewanella, and the genus Achromobacter with that of the normal individual-derived sample;
comparing an increase or decrease in content of extracellular vesicles derived from one or more bacteria selected from the group consisting of the order Bacillales, the order Rickettsiales, and the order 1025 with that of the chronic obstructive pulmonary disease patient-derived sample;
comparing an increase or decrease in content of extracellular vesicles derived from one or more bacteria selected from the group consisting of the family Staphylococcaceae, the family Nocardiaceae, and the family Rs-045 with that of the chronic obstructive pulmonary disease patient-derived sample;
comparing an increase or decrease in content of extracellular vesicles derived from one or more bacteria selected from the group consisting of the genus Alloiococcus, the genus Moraxella, the genus Staphylococcus, the genus Brevundimonas, the genus Enhydrobacter, the genus Comamonas, and the genus Rhodococcus with that of the chronic obstructive pulmonary disease patient-derived sample;
comparing an increase or decrease in content of extracellular vesicles derived from one or more bacteria selected from the group consisting of the phylum Bacteroidetes, the phylum Cyanobacteria, the phylum TM7, the phylum Fusobacteria, the phylum Thermi, the phylum Verrucomicrobia, the phylum Armatimonadetes, the phylum Acidobacteria, the phylum Gemmatimonadetes, and the phylum Chloroflexi with that of the asthma patient-derived sample;
comparing an increase or decrease in content of extracellular vesicles derived from one or more bacteria selected from the group consisting of the class Bacteroidia, the class Bacilli, the class Flavobacteriia, the class Sphingobacteriia, the class Alphaproteobacteria, the class Fusobacteriia, the class TM7-3, the class Deinococci, the class Verrucomicrobiae, the class Saprospirae, the class Chloroplast, the class Cytophagia, the class Fimbriimonadia, the class Chloracidobacteria, the class Thermomicrobia, the class Thermoleophilia, and the class Solibacteres with that of the asthma patient-derived sample;
comparing an increase or decrease in content of extracellular vesicles derived from one or more bacteria selected from the group consisting of the order YS2, the order Turicibacterales, the order Bifidobacteriales, the order Bacteroidales, the order Enterobacteriales, the order Rhodobacterales, the order Gemellales, the order Flavobacteriales, the order Caulobacterales, the order Neisseriales, the order Sphingobacteriales, the order Deinococcales, the order Pseudomonadales, the order Rhodocyclales, the order Xanthomonadales, the order Fusobacteriales, the order Actinomycetales, the order Sphingomonadales, the order Verrucomicrobiales, the order Saprospirales, the order Rhizobiales, the order Bacillales, the order Streptophyta, the order Cytophagales, the order Thermales, the order Fimbriimonadales, the order CW040, the order Rickettsiales, the order RB41, the order Alteromonadales, the order JG30-KF-CM45, the order 1025, the order Aeromonadales, and the order Solibacterales with that of the asthma patient-derived sample;
comparing an increase or decrease in content of extracellular vesicles derived from one or more bacteria selected from the group consisting of the family Helicobacteraceae, the family Bacteroidaceae, the family Turicibacteraceae, the family Veillonellaceae, the family Bifidobacteriaceae, the family Barnesiellaceae, the family Rikenellaceae, the family Clostridiaceae, the family Odoribacteraceae, the family Enterobacteriaceae, the family Porphyromonadaceae, the family Gemellaceae, the family Weeksellaceae, the family Carnobacteriaceae, the family Leptotrichiaceae, the family Moraxellaceae, the family Caulobacteraceae, the family Erythrobacteraceae, the family Hyphomicrobiaceae, the family Neisseriaceae, the family Sphingobacteriaceae, the family Deinococcaceae, the family Aerococcaceae, the family Bartonellaceae, the family Micrococcaceae, the family Flavobacteriaceae, the family Burkholderiaceae, the family Lactobacillaceae, the family Dietziaceae, the family Rhodocyclaceae, the family Xanthomonadaceae, the family Geodermatophilaceae, the family Actinomycetaceae, the family Methylobacteriaceae, the family Pseudomonadaceae, the family Corynebacteriaceae, the family Staphylococcaceae, the family Nocardioidaceae, the family Verrucomicrobiaceae, the family Sphingomonadaceae, the family Mycobacteriaceae, the family Tissierellaceae, the family Chitinophagaceae, the family Intrasporangiaceae, the family Propionibacteriaceae, the family Aurantimonadaceae, the family Planococcaceae, the family Fusobacteriaceae, the family Bradyrhizobiaceae, the family Nocardiaceae, the family Dermabacteraceae, the family Bacillaceae, the family Thermaceae, the family Ellin6075, the family Brevibacteriaceae, the family Microbacteriaceae, the family Rhodospirillaceae, the family Cytophagaceae, the family Fimbriimonadaceae, the family Dermacoccaceae, the family Chromatiaceae, the family Rhizobiaceae, the family Gordoniaceae, the family mitochondria, the family Pseudonocardiaceae, the family Exiguobacteraceae, the family Shewanellaceae, the family F16, the family Rs-045, and the family Aeromonadaceae with that of the asthma patient-derived sample; and
comparing an increase or decrease in content of extracellular vesicles derived from one or more bacteria selected from the group consisting of the genus Trabulsiella, the genus Enterobacter, the genus Veillonella, the genus Bifidobacterium, the genus Lachnospira, the genus Comamonas, the genus Bacteroides, the genus Turicibacter, the genus Sutterella, the genus Klebsiella, the genus SMB53, the genus Roseburia, the genus Dialister, the genus Ruminococcus, the genus Parabacteroides, the genus Butyricimonas, the genus Odoribacter, the genus Eubacterium, the genus Dorea, the genus Enhydrobacter, the genus Granulicatella, the genus Chryseobacterium, the genus Porphyromonas, the genus Coprococcus, the genus Peptoniphilus, the genus Microbispora, the genus Deinococcus, the genus Acinetobacter, the genus Aerococcus, the genus Actinomyces, the genus Brevundimonas, the genus Blastomonas, the genus Citrobacter, the genus Lactobacillus, the genus Stenotrophomonas, the genus Corynebacterium, the genus Pseudomonas, the genus Lautropia, the genus Akkermansia, the genus Staphylococcus, the genus Bacillus, the genus Sphingobacterium, the genus Anaerococcus, the genus Neisseria, the genus Leptotrichia, the genus Mycobacterium, the genus Kocuria, the genus Methylobacterium, the genus Propionibacterium, the genus Hymenobacter, the genus Sphingomonas, the genus Fusobacterium, the genus Brachybacterium, the genus Rhodococcus, the genus Micrococcus, the genus Kaistobacter, the genus Finegoldia, the genus Rubellimicrobium, the genus Brevibacterium, the genus Agrobacterium, the genus Dietzia, the genus Fimbriimonas, the genus Flavobacterium, the genus Dermacoccus, the genus Skermanella, the genus Novosphingobium, the genus Gordonia, the genus Rheinheimera, the genus Achromobacter, the genus Hydrogenophilus, the genus Thermus, the genus Exiguobacterium, the genus Shewanella, the genus Ralstonia, and the genus Alkanindiges with that of the asthma patient-derived sample.

3.-14. (canceled)

15. The method of claim 1, wherein the subject sample is blood.

16. The method of claim 15, wherein the blood is whole blood, serum, plasma, or blood mononuclear cells.

17. A method of diagnosing lung cancer, the method comprising the following processes:
(a) extracting DNA from extracellular vesicles isolated from a subject sample;
(b) performing polymerase chain reaction (PCR) on the extracted DNA using a pair of primers having SEQ ID NO:1 and SEQ ID NO: 2; and
(c) comparing an increase or decrease in content of bacteria-derived extracellular vesicles of the subject sample with that of a normal individual-derived sample, a chronic obstructive pulmonary disease patient-derived sample, or an asthma patient-derived sample through sequencing of a product of the PCR.

18. The method of claim 17, wherein the comparing comprises at least one selected from the group consisting of:
comparing an increase or decrease in content of extracellular vesicles derived from one or more bacteria selected from the group consisting of the phylum Acidobacteria and the phylum Chloroflexi with that of the normal individual-derived sample;
comparing an increase or decrease in content of extracellular vesicles derived from one or more bacteria selected from the group consisting of the class Thermomicrobia and the class Solibacteres with that of the normal individual-derived sample;
comparing an increase or decrease in content of extracellular vesicles derived from one or more bacteria selected from the group consisting of the order Turicibacterales, the order Rickettsiales, the order Alteromonadales, the order RF32, the order Thermales, the order JG30-KF-CM45, the order I025, the order Solibacterales, and the order Aeromonadales with that of the normal individual-derived sample;
comparing an increase or decrease in content of extracellular vesicles derived from one or more bacteria selected from the group consisting of the family Turicibacteraceae, the family Clostridiaceae, the family S24-7, the family Rhizobiaceae, the family mitochondria, the family F16, the family Gordoniaceae, the family Rhodospirillaceae, the family Thermaceae, the family Shewanellaceae, the family Ellin6075, the family Rs-045, and the family Aeromonadaceae with that of the normal individual-derived sample;
comparing an increase or decrease in content of extracellular vesicles derived from one or more bacteria selected from the group consisting of the genus Chromohalobacter, the genus Geobacillus, the genus Proteus, the genus Megamonas, the genus Moraxella, the genus Alloiococcus, the genus Turicibacter, the genus SMB53, the genus Veillonella, the genus Peptomphilus, the genus Comamonas, the genus Hymenobacter, the genus Citrobacter, the genus Novosphingobium, the genus Gordonia, the genus Aerococcus, the genus Thermus, the genus Shewanella, and the genus Achromobacter with that of the normal individual-derived sample;
comparing an increase or decrease in content of extracellular vesicles derived from one or more bacteria selected from the group consisting of the order Bacillales, the order Rickettsiales, and the order I025 with that of the chronic obstructive pulmonary disease patient-derived sample;
comparing an increase or decrease in content of extracellular vesicles derived from one or more bacteria selected from the group consisting of the family Staphylococcaceae, the family Nocardiaceae, and the family Rs-045 with that of the chronic obstructive pulmonary disease patient-derived sample;
comparing an increase or decrease in content of extracellular vesicles derived from one or more bacteria selected from the group consisting of the genus Alloiococcus, the genus Moraxella, the genus Staphylococcus, the genus Brevundimonas, the genus Enhydrobacter, the genus Comamonas, and the genus Rhodococcus with that of the chronic obstructive pulmonary disease patient-derived sample;
comparing an increase or decrease in content of extracellular vesicles derived from one or more bacteria selected from the group consisting of the phylum Bacteroidetes, the phylum Cyanobacteria, the phylum TM7, the phylum Fusobacteria, the phylum Thermi, the phylum Verrucomicrobia, the phylum Armatimonadetes, the phylum Acidobacteria, the phylum Gemmatimonadetes, and the phylum Chloroflexi with that of the asthma patient-derived sample;
comparing an increase or decrease in content of extracellular vesicles derived from one or more bacteria selected from the group consisting of the class Bacteroidia, the class Bacilli, the class Flavobacteriia, the class Sphingobacteriia, the class Alphaproteobacteria, the class Fusobacteriia, the class TM7-3, the class Deinococci, the class Verrucomicrobiae, the class Saprospirae, the class Chloroplast, the class Cytophagia, the class Fimbriimonadia, the class Chloracidobacteria, the class Thermomicrobia, the class Thermoleophilia, and the class Solibacteres with that of the asthma patient-derived sample;
comparing an increase or decrease in content of extracellular vesicles derived from one or more bacteria selected from the group consisting of the order YS2, the order Turicibacterales, the order Bifidobacteriales, the order Bacteroidales, the order Enterobacteriales, the order Rhodobacterales, the order Gemellales, the order Flavobacteriales, the order Caulobacterales, the order Neisseriales, the order Sphingobacteriales, the order Deinococcales, the order Pseudomonadales, the order Rhodocyclales, the order Xanthomonadales, the order Fusobacteriales, the order Actinomycetales, the order Sphingomonadales, the order Verrucomicrobiales, the order Saprospirales, the order Rhizobiales, the order Bacillales, the order Streptophyta, the order Cytophagales, the order Thermales, the order Fimbriimonadales, the order CW040, the order Rickettsiales, the order RB41, the order Alteromonadales, the order JG30-KF-CM45, the order 1025, the order Aeromonadales, and the order Solibacterales with that of the asthma patient-derived sample;
comparing an increase or decrease in content of extracellular vesicles derived from one or more bacteria selected from the group consisting of the family Helicobacteraceae, the family Bacteroidaceae, the family Turicibacteraceae, the family Veillonellaceae, the family Bifidobacteriaceae, the family Barnesiellaceae, the family Rikenellaceae, the family Clostridiaceae, the family Odoribacteraceae, the family Enterobacteriaceae, the family Porphyromonadaceae, the family Gemellaceae, the family Weeksellaceae, the family Carnobacteriaceae, the family Leptotrichiaceae, the family Moraxellaceae, the family Caulobacteraceae, the family Erythrobacteraceae, the family Hyphomicrobiaceae, the family Neisseriaceae, the family Sphingobacteriaceae, the family Deinococcaceae, the family Aerococcaceae, the family Bartonellaceae, the family Micrococcaceae, the family Flavobacteriaceae, the family Burkholderiaceae, the family Lactobacillaceae, the family Dietziaceae, the family Rhodocyclaceae, the family Xanthomonadaceae, the family Geodermatophilaceae, the family Actinomycetaceae, the family Methylobacteriaceae, the family Pseudomonadaceae, the family Corynebacteriaceae, the family Staphylococcaceae, the family Nocardioidaceae, the family Verrucomicrobiaceae, the family Sphingomonadaceae, the family Mycobacteriaceae, the family Tissierellaceae, the family Chitinophagaceae, the family Intrasporangiaceae, the family Propionibacteriaceae, the family Aurantimonadaceae, the family Planococcaceae, the family Fusobacteriaceae, the family Bradyrhizobiaceae, the family Nocardiaceae, the family Dermabacteraceae, the family Bacillaceae, the family Thermaceae, the family Ellin6075, the family Brevibacteriaceae, the family Microbacteriaceae, the family Rhodospirillaceae, the family Cytophagaceae, the family Fimbriimonadaceae, the family Dermacoccaceae, the family Chromatiaceae, the family Rhizobiaceae, the family Gordoniaceae, the family mitochondria, the family Pseudonocardiaceae, the family Exiguobacteraceae, the family Shewanellaceae, the family F16, the family Rs-045, and the family Aeromonadaceae with that of the asthma patient-derived sample; and
comparing an increase or decrease in content of extracellular vesicles derived from one or more bacteria selected from the group consisting of the genus Trabulsiella, the genus Enterobacter, the genus Veillonella, the genus Bifidobacterium, the genus Lachnospira, the genus Comamonas, the genus Bacteroides, the genus Turicibacter, the genus Sutterella, the genus Klebsiella, the genus SMB53, the genus Roseburia, the genus Dialister, the genus Ruminococcus, the genus Parabacteroides, the genus Butyricimonas, the genus Odoribacter, the genus Eubacterium, the genus Dorea, the genus Enhydrobacter, the genus Granulicatella, the genus Chryseobacterium, the genus Porphyromonas, the genus Coprococcus, the genus Peptoniphilus, the genus Microbispora, the genus Deinococcus, the genus Acinetobacter, the genus Aerococcus, the genus Actinomyces, the genus Brevundimonas, the genus Blastomonas, the genus Citrobacter, the genus Lactobacillus, the genus Stenotrophomonas, the genus Corynebacterium, the genus Pseudomonas, the genus Lautropia, the genus Akkermansia, the genus Staphylococcus, the genus Bacillus, the genus Sphingobacterium, the genus Anaerococcus, the genus Neisseria, the genus Leptotrichia, the genus Mycobacterium, the genus Kocuria, the genus Methylobacterium, the genus Propionibacterium, the genus Hymenobacter, the genus Sphingomonas, the genus Fusobacterium, the genus Brachybacterium, the genus Rhodococcus, the genus Micrococcus, the genus Kaistobacter, the genus Finegoldia, the genus Rubellimicrobium, the genus Brevibacterium, the genus Agrobacterium, the genus Dietzia, the genus Fimbriimonas, the genus Flavobacterium, the genus Dermacoccus, the genus Skermanella, the genus Novosphingobium, the genus Gordonia, the genus Rheinheimera, the genus Achromobacter, the genus Hydrogenophilus, the genus Thermus, the genus Exiguobacterium, the genus Shewanella, the genus Ralstonia, and the genus Alkanindiges with that of the asthma patient-derived sample.

19.-30. (canceled)

31. The method of claim 17, wherein the subject sample is blood.

32. The method of claim 31, wherein the blood is whole blood, serum, plasma, or blood mononuclear cells.
